(19) United States
(12) Patent Application Publication
(10) Pub. No.: US 2005/0124498 A1
(43) Pub. Date: Jun. 9, 2005

(54) Halobenzyl substituted phenylaceto esters and amides and use thereof for prevention of harmful organisms

(76) Inventors: Fritz Maurer, Lauffen (DE); Christiane Boie, Leichlingen (DE); Herbert Gayer, Monheim (DE); Ulrich Heinemann, Leichlingen (DE); Bernd-Wieland Kruger, Bergisch Gladbach (DE); Ulrike Wachendorff-Neumann, Neuwied (DE); Astrid Mauler-Machnik, Leichlingen (DE); Peter Losel, Leverkusen (DE)

Correspondence Address: BAYER CROPSCIENCE LP;Patent Department, 100 BAYER ROAD, PITTSBURGH, PA, 15205-9741, US

(21) Appl. No.: 10/506,148

(22) PCT Filed: Feb. 17, 2003
(86) PCT No.: PCT/EP03/01565

(30) Foreign Application Priority Data
Mar. 1, 2002 (DE) 10209145.5

(51) Int. Cl. A01N 43/50; C07D 273/04; C07C 251/48

(52) U.S. Cl. 504/277; 544/309; 544/67; 560/168; 560/35

(57) ABSTRACT

The invention relates to novel halobenzenes, to a plurality of processes for their preparation and to their use for controlling harmful organisms, and also to novel intermediates and processes for their preparation.

The invention relates to novel halobenzenes, to a plurality of processes for their preparation and to their use for controlling harmful organisms, and also to novel intermediates and processes for their preparation.

Certain halo-substituted benzene derivatives having a substitution pattern similar to that of the novel compounds described below, and their use as agents for controlling harmful organisms have already been disclosed (compare, for example, WO 95-04728, WO 98-21189, EP 460525, EP 398692, EP 865424, WO 95-18789, EP 253213, EP 382375). However, in particular at low application rates and concentrations, the action of these prior-art compounds is not entirely satisfactory in all areas of use.

This invention now provides the novel halobenzenes of the general formula (I) 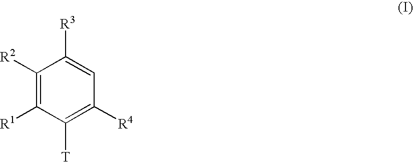
in which
R¹ represents one of the groupings 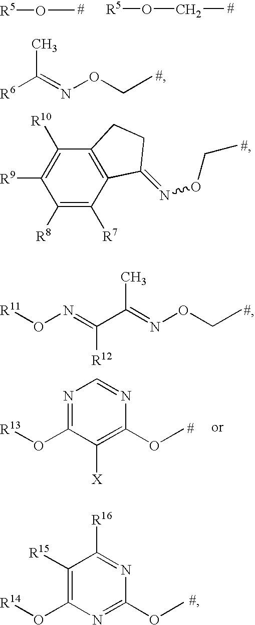
   in which
   R⁵ represents optionally substituted aryl,
   R⁶ represents optionally substituted aryl,
   R⁷,R⁸,R⁹ and R¹⁰ are identical or different, and independently of one another represent hydrogen, halogen, optionally substituted alkyl or optionally substituted alkoxy,
   R¹¹ represents optionally substituted alkyl,
   R¹² represents in each case optionally substituted alkyl, alkoxy or aryl,
   R¹³ represents in each case optionally substituted alkyl or aryl,
   R¹⁴ represents optionally substituted aryl,
   R¹⁵ and R¹⁶ are identical or different and dependently of one another represent hydrogen, halogen or alkyl,
   X represents hydrogen or halogen,
R², R³ and R⁴ are identical or different and independently of one another represent hydrogen or halogen, where however at least one of the radicals R², R³ and R⁴ represents halogen, and
T represents a grouping

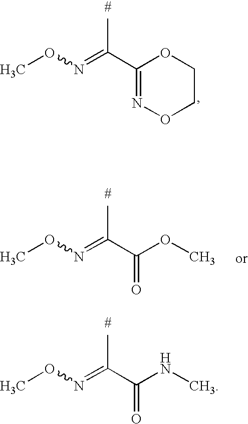

In the definitions, the saturated or unsaturated hydrocarbon chains, such as alkyl, alkanediyl, alkenyl or alkynyl, are in each case straight-chain branched, including in combination with heteroatoms, such as, for example, in alkoxy, alkylthio or alkylamino. Unless indicated otherwise, preference is given to hydrocarbon chains having 1 to 6 carbon atoms.

Halogen generally represents fluorine, chlorine, bromine or iodine, preferably fluorine, chlorine or bromine, in particular fluorine or chlorine.

Aryl represents aromatic mono- or polycyclic hydrocarbon rings, such as, for example, phenyl, naphthyl, anthranyl, phenanthryl, preferably phenyl or naphthyl, in particular phenyl.

Furthermore, it has been found that the novel halobenzenes of the general formula (I) are obtained when
a) a hydroxyhalophenyl derivative of the formula (II) 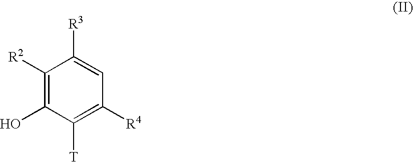
   in which
   R², R³, R⁴ and T are as defined above
   is reacted with a phenoxypyrimidine of the general formula (III) 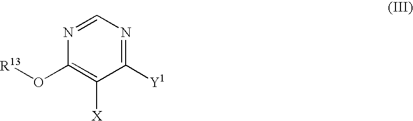
   in which
   R¹³ and X are as defined above and
   Y¹ represents halogen
   or an alkoxypyrimidine of the general formula (IV) 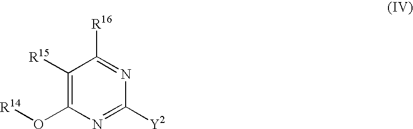
   in which
   R¹⁴, R¹⁵ and R¹⁶ are as defined above and
   Y² represents alkysulphonyl or arylsulphonyl,
   if appropriate in the presence of a diluent, if appropriate in the presence of an acid acceptor and if appropriate in the presence of a catalyst, or when
b) a 4-phenoxypyrimidine of the general formula (V) 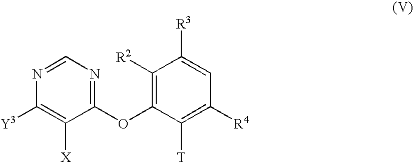
   in which
   R², R³, R⁴, T and X are as defined above and
   Y³ represents halogen
   is reacted with a phenol of the general formula (VI)
      R¹³—OH (VI)
   in which
   R¹³ is as defined above
   if appropriate in the presence of a diluent, if appropriate in the presence of an acid acceptor and if appropriate in the presence of a catalyst, or when
c) a hydroxyhalophenyl derivative of the formula (II) is reacted with a diaryliodonium halide of the general formula (VII) 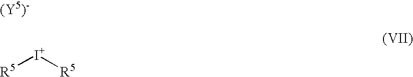
   in which
   R⁵ is as defined above and
   Y⁵ represents halogen,
   if appropriate in the presence of a diluent and if appropriate in the presence of an acid acceptor, or when
d) a 2-(2-halomethylphenyl)-2-methoxyimino derivative of the formula (VIII) 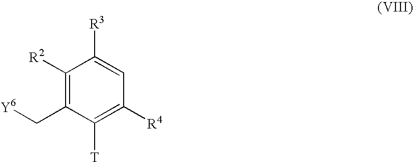
   in which
   R², R³, R⁴ and T are as defined above and
   Y⁶ represents halogen
   is reacted with an oxime of the formula (IX) 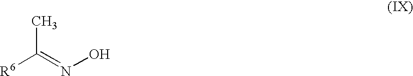
   in which
   R⁶ is as defined above
   or with a benzofuranone oxime of the formula (X) 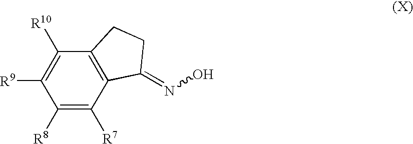
   in which
   R⁷, R⁸, R⁹ and R¹⁰ are as defined above
   or with a bisoxime of the formula (XI) 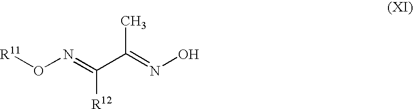
   in which
   R¹¹ and R¹² are as defined above
   or with a phenol of the formula (XII)
      R⁵—OH (XII)
   in which
   R⁵ is as defined above
   if appropriate in the presence of a diluent and if appropriate in the presence of an acid acceptor, or when
e) a halobenzene of the general formula (I-a) 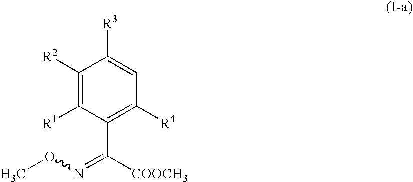
   in which
   R¹, R², R³ and R⁴ are as defined above
   is reacted with methylamine, if appropriate in the presence of a diluent, or when
f) a halobenzene of the general formula (I-a) is, in a first step, reacted with hydroxylamine or a salt thereof, if appropriate in the presence of an acid acceptor and if appropriate in the presence of a diluent, and, without isolation of the product of the first step, reacted in a second step with dibromoethane, if appropriate in the presence of an acid acceptor and if appropriate in the presence of a diluent.

Finally, it has been found that the novel halobenzenes of the general formula (I) show strong action against harmful organisms, in particular a very strong fungicidal action.

If appropriate, the compounds according to the invention can be present as mixtures of different possible isomeric forms, in particular of stereoisomers, such as, for example, E and Z. What is claimed are both the E and the Z isomers and any mixtures of these isomers.

The invention preferably provides compounds of the formula (I) in which
R¹ represents in each case optionally substituted phenoxy or phenoxymethyl or represents one of the groupings 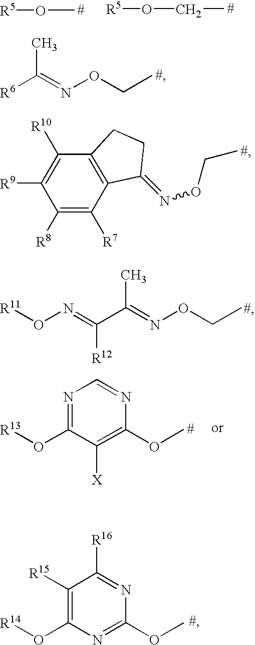
   in which
   R⁵ represents optionally substituted phenyl,
   R⁶ represents optionally substituted phenyl,
   R⁷, R⁸,R⁹and R¹⁰ are identical or different and independently of one another represent hydrogen, optionally halogen-substituted alkyl or optionally halogen-substituted alkoxy having in each case 1 to 4 carbon atoms, fluorine, chlorine, bromine or iodine,
   R¹¹ represents optionally halogen-substituted alkyl having 1 to 4 carbon atoms,
   R¹² represents in each case optionally halogen-substituted alkyl or alkoxy having 1 to 6 carbon atoms or optionally substituted phenyl,
   R¹³ represents in each case optionally halogen-substituted alkyl having 1 to 6 carbon atoms or optionally substituted phenyl,
   R¹⁴ represents optionally halogen-substituted alkyl having 1 to 6 carbon atoms,
   R¹⁵ and R¹⁶ are identical or different and independently of one another represent hydrogen, fluorine, chlorine, bromine or alkyl having 1 to 4 carbon atoms,
   X represents hydrogen, fluorine, chlorine, bromine or iodine,
R², R³ and R⁴ are identical or different and independently of one another represent hydrogen, fluorine, chlorine, bromine or iodine, where however at least one of the radicals R², R³ and R⁴ represents fluorine, chlorine, bromine or iodine, and
   T represents a grouping

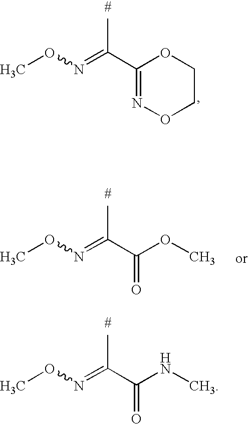

The substituents of the abovementioned phenyl, phenoxy and phenoxymethyl radicals are preferably selected from the following list:
halogen, cyano, nitro, amino, hydroxyl, formyl, carboxyl, carbamoyl, thiocarbamoyl; in each case straight-chain or branched alkyl, hydroxyalkyl, oxoalkyl, alkoxy, alkoxyalkyl, alkylthioalkyl, dialkoxyalkyl, alkylthio, alkylsulphinyl or alkylsulphonyl having in each case 1 to 8 carbon atoms;
in each case straight-chain or branched alkenyl or alkenyloxy having in each case 2 to 6 carbon atoms;
in each case straight-chain or branched haloalkyl, haloalkoxy, haloalkylthio, haloalkylsulphinyl or haloalkylsulphonyl having in each case 1 to 6 carbon atoms and 1 to 13 identical or different halogen atoms;
in each case straight-chain or branched haloalkenyl or haloalkenyloxy having in each case 2 to 6 carbon atoms and 1 to 11 identical or different halogen atoms;
in each case straight-chain or branched alkylamino, dialkylamino,
alkylcarbonyl, alkylcarbonyloxy, alkoxycarbonyl, alkylaminocarbonyl, dialkylaminocarbonyl, arylalkylaminocarbonyl, dialkylaminocarbonyloxy, alkenylcarbonyl or alkynylcarbonyl having 1 to 6 carbon atoms in the respective hydrocarbon chains;
cycloalkyl or cycloalkyloxy having in each case 3 to 6 carbon atoms;
in each case doubly attached alkylene having 3 or 4 carbon atoms, oxyalkylene having 2 or 3 carbon atoms or dioxyalkylene having 1 or 2 carbon atoms, each of which radicals is optionally mono- to tetrasubstituted by identical or different substituents from the group consisting of fluorine, chlorine, oxo, methyl, trifluoromethyl and ethyl;
or a grouping 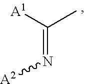
in which
   A¹ represents hydrogen, hydroxyl or alkyl having 1 to 4 carbon atoms or cycloalkyl having 1 to 6 carbon atoms and
   A² represents hydroxyl, amino, methylamino, phenyl, benzyl or represents in each case optionally cyano-, hydroxyl-, alkoxy-, alkylthio-, alkylamino-, dialkylamino- or phenyl-substituted alkyl or alkoxy having 1 to 4 carbon atoms, or represents alkenyloxy or alkynyloxy having in each case 2 to 4 carbon atoms.

The invention relates in particular to compounds of the formula (I) in which
R¹ represents in each case optionally substituted phenoxy or phenoxymethyl or represents one of the groupings 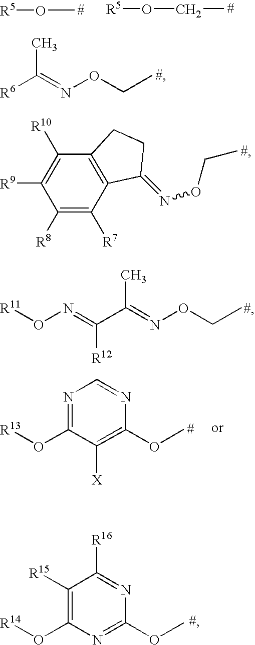
   in which
   R⁵ represents optionally substituted phenyl,
   R⁶ represents optionally substituted phenyl,
   R⁷, R⁸, R⁹ and R¹⁰ are identical or different and independently of one another represent hydrogen, methyl, ethyl, trifluoromethyl, methoxy, ethoxy, trifluoromethoxy, fluorine, chlorine, bromine or iodine,
   R¹¹ represents methyl, ethyl, n- or i-propyl, n-, i-, s- or t-butyl,
   R¹² represents methyl, ethyl, n- or i-propyl, n-, i-, s- or t-butyl, methoxy, ethoxy, n- or i-propoxy, n-, i-, s- or t-butoxy or optionally substituted phenyl,
   R¹³ represents methyl, ethyl, n- or i-propyl, n-, i-, s- or t-butyl, pentyl, hexyl or optionally substituted phenyl,
   R¹⁴ represents in each case optionally fluorine- or chlorine-substituted methyl, ethyl, n- or i-propyl, n-, i-, s- or t-butyl, pentyl, hexyl,
   R¹⁵ and R¹⁶ are identical or different and independently of one another represent hydrogen, fluorine, chlorine, bromine, methyl or ethyl,
   X represents hydrogen, fluorine, chlorine, bromine or iodine,
R², R³ and R⁴ are identical or different and independently of one another represent hydrogen, fluorine or chlorine, where however at least one of the radicals R², R³ and R⁴ represents fluorine or chlorine, and
T represents a grouping

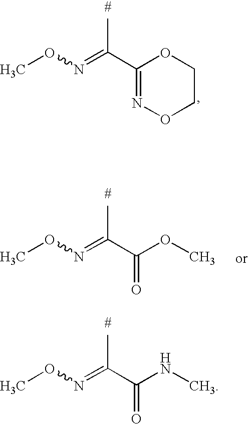

The substituents of the abovementioned phenyl radicals are preferably selected from the list below:
fluorine, chlorine, bromine, iodine, cyano, nitro, amino, hydroxyl, formyl, carboxy, carbamoyl, thiocarbamoyl,
methyl, ethyl, n- or i-propyl, n-, i-, s- or t-butyl, 1-, 2-, 3-, neo-pentyl, 1-, 2-, 3-, 4-(2-methylbutyl), 1-, 2-, 3-hexyl, 1-, 2-, 3-, 4-, 5-(2-methylpentyl), 1-, 2-, 3-(3-methylpentyl), 2-ethylbutyl, 1-, 3-, 4-(2,2-dimethylbutyl), 1-, 2-(2,3-dimethylbutyl), hydroxymethyl, hydroxyethyl, 3-oxobutyl, methoxymethyl, dimethoxymethyl,
methoxy, ethoxy, n- or i-propoxy, methoxymethyl, ethoxymethyl,
methylthio, ethylthio, n- or i-propylthio, methylsulphinyl, ethylsulphinyl, methylsulphonyl or ethylsulphonyl, methylthiomethyl, ethylthiomethyl, vinyl, allyl, 2-methylallyl, propen-1-yl, crotonyl, propargyl, vinyloxy, allyloxy, 2-methylallyloxy, propen-1-yloxy, crotonyloxy, propargyloxy;
trifluoromethyl, trifluoroethyl,
difluoromethoxy, trifluoromethoxy, difluorochloromethoxy, trifluoroethoxy, difluoromethylthio, trifluoromethylthio, difluorochloromethylthio, trifluoromethylsulphinyl or trifluoromethylsulphonyl,
methylamino, ethylamino, n- or i-propylamino, dimethylamino, diethylamino,
acetyl, propionyl, methoxycarbonyl, ethoxycarbonyl, methylaminocarbonyl, ethylaminocarbonyl, dimethylaminocarbonyl, diethylaminocarbonyl, dimethylaminocarbonyloxy, diethylaminocarbonyloxy, benzylaminocarbonyl, acryloyl, propioloyl,
cyclopentyl, cyclohexyl,
in each case doubly attached propanediyl, ethyleneoxy, methylenedioxy, ethylenedioxy, each of which is optionally mono- to tetra-substituted by identical or different substituents from the group consisting of fluorine, chlorine, oxo, methyl and trifluoromethyl,
or a grouping 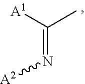
where
A¹ represents hydrogen, methyl or hydroxyl and
A² represents hydroxyl, methoxy, ethoxy, amino, methylamino, phenyl, benzyl or hydroxyethyl.

The general or preferred radical definitions given above apply both to the end products of the formula (I) and, correspondingly, to the starting materials or intermediates required in each case for the preparation.

The particular radical definitions given in the respective combinations or preferred combinations of radicals individually for these radicals can, independently of the given combination, also be replaced by any radical definitions of other preferred ranges.

The process a) according to the invention can be illustrated by the reaction equations below:

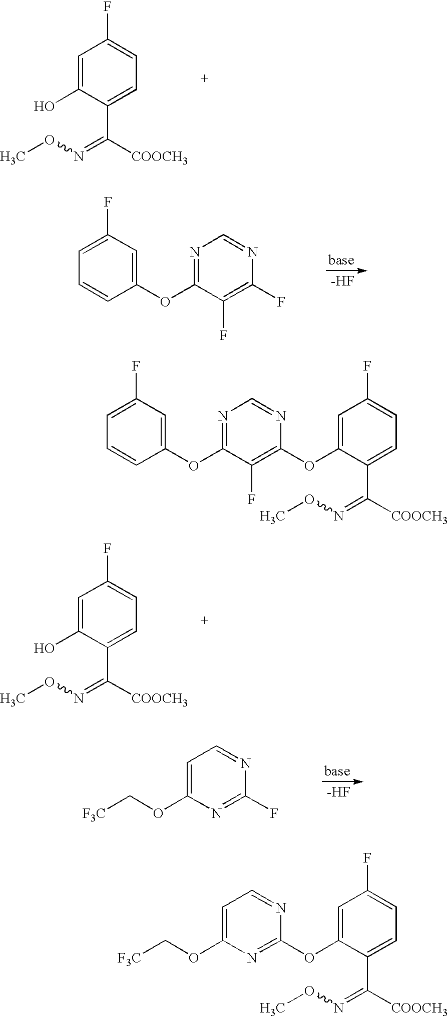

Formula (II) provides a general definition of the hydroxyhalophenyl derivatives required as starting materials for carrying out the process a) according to the invention. In this formula (II), R², R³, R⁴ and T preferably or in particular have those meanings which have already been given in connection with the description of the compounds of the formula (I) according to the invention as being preferred or as being particularly preferred for R², R³, R⁴ and T.

The starting materials of the formula (II) are novel and also form part of the subject-matter of the present application.

They are obtained (process g) when a ketoester of the general formula (XIII) 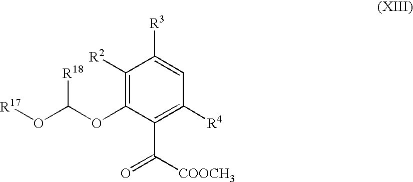
in which
R², R³ and R⁴ are as defined above and
R¹⁷ represents alkyl,
R¹⁸ represents hydrogen or alkyl or
R¹⁷ and R¹⁸ together with the atoms to which they are attached form a six-membered ring
or a hydroxyphenylglyoxylic acid ester of the general formula (XIV) 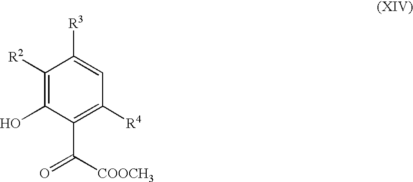
in which
R², R³ and R⁴ are as defined above
is reacted with methoxyamine hydrochloride, if appropriate in the presence of a diluent,
or when (process h) a hydroxyhalophenyl derivative of the formula (II-a) 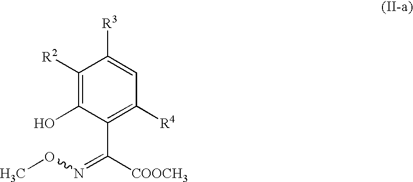
in which
R², R³ and R⁴ are as defined above
is reacted with methylamine, if appropriate in the presence of a diluent.

The process g) according to the invention can be illustrated by the following reaction equations:

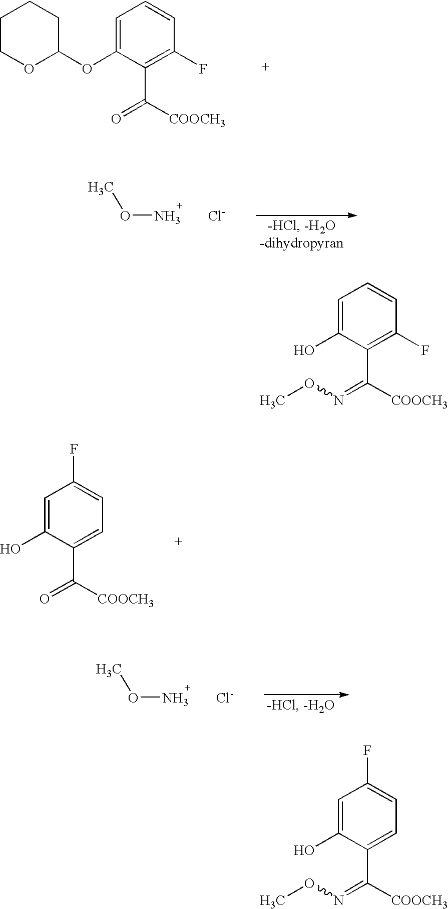

The formula (XIII) provides a general definition of the ketoesters required as starting materials for carrying out the process g) according to the invention. In this formula (XIII), R², R³ and R⁴ preferably or in particular have those meanings which have already been given in connection with the description of compounds of the formula (I) according to the invention as being preferred or as being particularly preferred for R², R³ and R⁴. R¹⁷ represents alkyl, preferably methyl or ethyl. R¹⁸ represents hydrogen or alkyl, preferably hydrogen, methyl or ethyl Together with the atoms to which they are attached, R¹⁷ and R¹⁸ also represent a six-membered ring, preferably a tetrahydropyran ring.

The keto esters of the formula (XIII) are novel and also form part of the subject-matter of the present invention. They are obtained (process i) when a ketal of the general formula (XV) 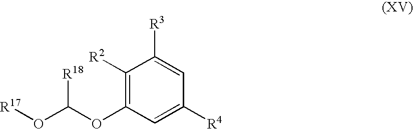
in which
R², R³, R¹⁷ and R¹⁸ are as defined above
is reacted with dimethyl oxalate, if appropriate in the presence of a diluent and if appropriate in the presence of a strong base.

The process i) according to the invention can be illustrated by the reaction equation below:

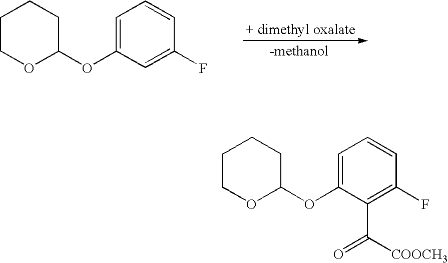

The formula (XV) provides a general definition of the ketals required as starting materials for carrying out the process i) according to the invention. In this formula (XV), R², R³, R¹⁷ and R¹⁸ preferably or in particular have those meanings which have already been given in connection with the description of the compounds of the formulae (I) and (XIII) according to the invention as being preferred or as being particularly preferred for R², R³, R¹⁷ and R¹⁸.

The compounds of the formula (XV) are known or can be prepared by known processes (compare, for example, J. Amer. Chem. Soc. 119; 6; 1997; 1208-1216).

Dimethyl oxalate, which is furthermore required for carrying out the process i) according to the invention, is a generally known laboratory chemical.

The formula (XIV) provides a general definition of the hydroxyphenylglyoxylic acid esters alternatively required as starting materials for carrying out the process g) according to the invention. In this formula (XIV), R², R³ and R⁴ preferably or in particular have those meanings which have already been given in connection with the description of the compounds of the formula (I) according to the invention as being preferred or as being particularly preferred for R², R³ and R⁴.

The hydroxyphenylglyoxylic acid esters of the formula (XIV) are novel and also form part of the subject-matter of the present application. They are obtained (process j) when fluorophenols of the general formula (XVI) 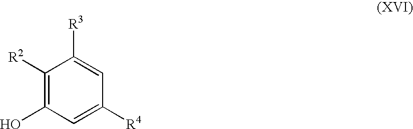
in which
R², R³ and R⁴ are as defined above are reacted with methyl oxalyl chloride, if appropriate in the presence of a diluent and if appropriate in the presence of a Lewis acid, or when process k) the ketals of the formula (XV) described above are hydrolysed, if appropriate in the presence of a diluent and if appropriate in the presence of an acid.

The process j) according to the invention can be illustrated by the reaction equation below:

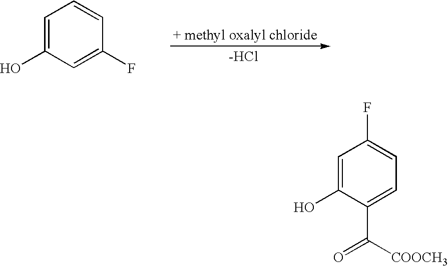

The formula (XVI) provides a general definition of the fluorophenols required as starting materials for carrying out the process j) according to the invention. In this formula (XVI), R², R³ and R⁴ preferably or in particular have those meanings which have already been given in connection with the description of the compounds of the formula (I) according to the invention as being preferred or as being particularly preferred for R², R³ and R⁴.

The fluorophenols of the formula (XVI) and methyl oxalyl chloride, furthermore required for carrying out the process j) according to the invention, are commercial laboratory chemicals.

The process k) according to the invention can be illustrated by the reaction equation below:

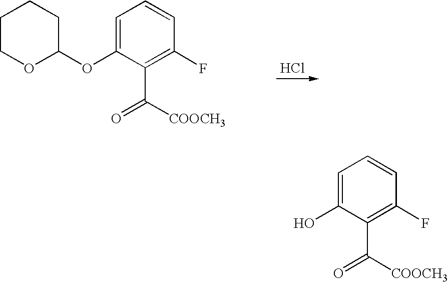

The ketals of the formula (XV) required as starting materials for carrying out the process k) according to the invention have already been described above, in connection with the description of the process g) according to the invention.

Hydroxylamine and its salts, furthermore required as starting materials for carrying out the process g) according to the invention, are commercial laboratory chemicals.

The process h) according to the invention can be illustrated by the following reaction equation:

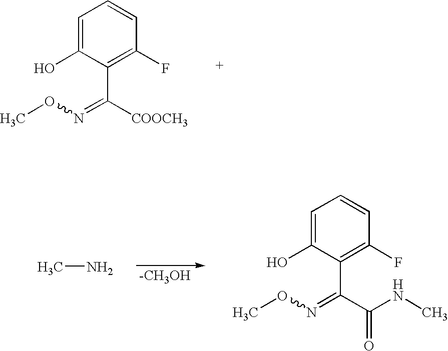

The formula (II-a) provides a general definition of the hydroxyhalophenyl derivatives required as starting materials for carrying out the process h) according to the invention. In this formula (II-a), R², R³ and R⁴ preferably or in particular have those meanings which have already been given in connection with the description of the compounds of the formula (I) according to the invention as being preferred or as being particularly preferred for R², R³ and R⁴.

The hydroxyhalophenyl derivatives of the formula (II-a) are compounds according to the invention and can be prepared by process g).

Methylamine, furthermore required as starting material for carrying out the process h) according to the invention, is a commerical laboratory chemical.

The formula (III) provides a general definition of the phenoxypyrimidines furthermore required as starting materials for carrying out the process a) according to the invention. In this formula (III), R¹³ and X preferably or in particular have those meanings which have already been given in connection with the description of the compounds of the formula (I) according to the invention as being preferred or as being particularly preferred for R¹³ and X. Y¹ represents halogen, preferably fluorine or chlorine.

The starting materials of the formula (III) are known and/or can be prepared by known methods (compare, for example, WO 97-27189).

The formula (IV) provides a general definition of the alkoxypyrimidines furthermore alternatively suitable as starting materials for carrying out the process a) according to the invention. In this formula (IV), R¹⁴, R¹⁵ and R¹⁶ preferably or in particular have those meanings which have already been given in connection with the description of the compounds of the formula (I) according to the invention as being preferred or as being particularly preferred for R¹⁴, R¹⁵ and R¹⁶. Y² represents alkylsulphonyl or arylsulphonyl, preferably methylsulphonyl or tolylsulphonyl.

The starting materials of the formula (IV) are known and/or can be prepared by known methods (compare, for example, WO 95-24396).

The process b) according to the invention can be illustrated by the following reaction equation:

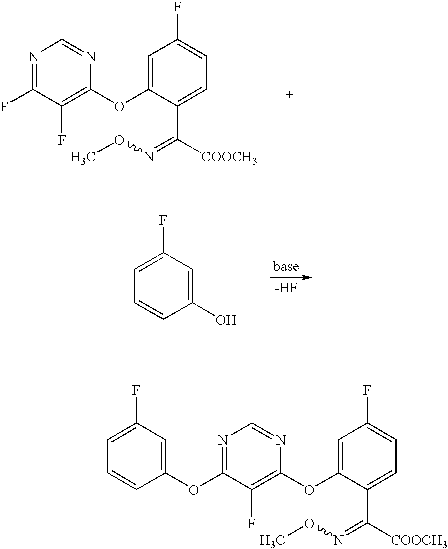

The formula (V) provides a general definition of the 4-phenoxypyrimidines required as starting materials for carrying out the process b) according to the invention. In this formula (V), R², R³, R⁴, T and X preferably or in particular have those meanings which have already been given in connection with the description of the compounds of the formula (I) according to the invention as being preferred or as being particularly preferred for R², R³, R⁴, T and X. Y³ represents halogen, preferably fluorine or chlorine.

The starting materials of the formula (V) are novel and also form part of the subject-matter of the present application. They are obtained when (process 1) halopyrimidines of the formula (XVII) 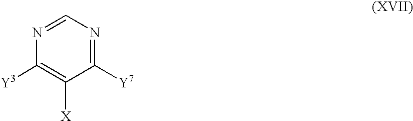
in which
Y³ and X are as defined above and
Y⁷ represents halogen
are reacted with a hydroxyhalophenyl derivative of the formula (II),
if appropriate in the presence of a diluent, if appropriate in the presence of an acid acceptor and if appropriate in the presence of a catalyst.

The process 1) according to the invention can be illustrated by the following reaction equation:

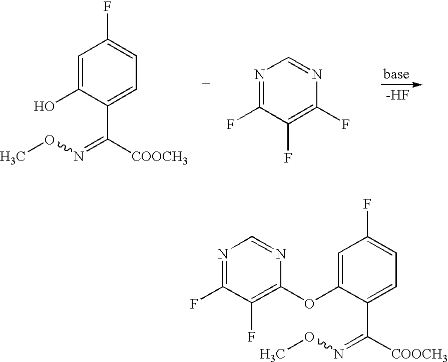

The formula (XVII) provides a general definition of the halopyrimidines required as starting materials for carrying out the process 1) according to the invention. In this formula (XVII), Y³ and X preferably or in particular have those meanings which have already been given in connection with the description of the compounds of the formula (V) according to the invention as being preferred or as being particularly preferred for Y³ and X. H⁷ represents halogen, preferably fluorine or chlorine.

The halopyrimidines of the formula (XVII) are known and can be prepared by known processes (compare, for example, WO 97-27189 or WO 98-41513).

The hydroxyhalophenyl derivatives of the formula (II) furthermore required as starting materials for carrying out the process 1) according to the invention have already been described above, in connection with the description of the process a) according to the invention.

The formula (VI) provides a general definition of the phenols furthermore required as starting materials for carrying out the process b) according to the invention. In this formula (VI), R¹³ preferably or in particular has that meaning which has already been given in connection with the description of the compounds of the formula (I) according to the invention as being preferred or as being particularly preferred for R¹³.

The phenols of the formula (VI) are generally known chemicals for synthesis.

The process c) according to the invention can be illustrated by the following reaction equation:

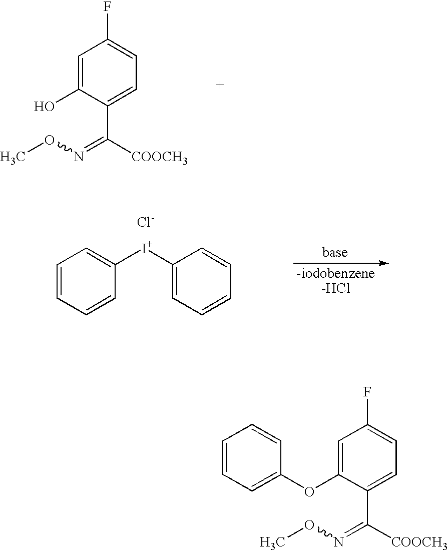

The hydroxyhalophenyl derivatives of the formula (II) required as starting materials for carrying out the process c) according to the invention have already been described above, in connection with the description of the process according to the invention.

The formula (VII) provides a general definition of the diaryliodonium halides furthermore required as starting materials for carrying out the process c) according to the invention. In this formula (VII), R⁵ preferably or in particular has that meaning which has already been given in connection with the description of the compounds of the formula (I) according to the invention as being preferred or as being particularly preferred for R⁵.

The diaryliodonium halides of the formula (VII) are known or can be prepared by known methods (compare, for example, Synthesis (1995), (8), 1027-3).

The process d) according to the invention can be illustrated by the following reaction equations:

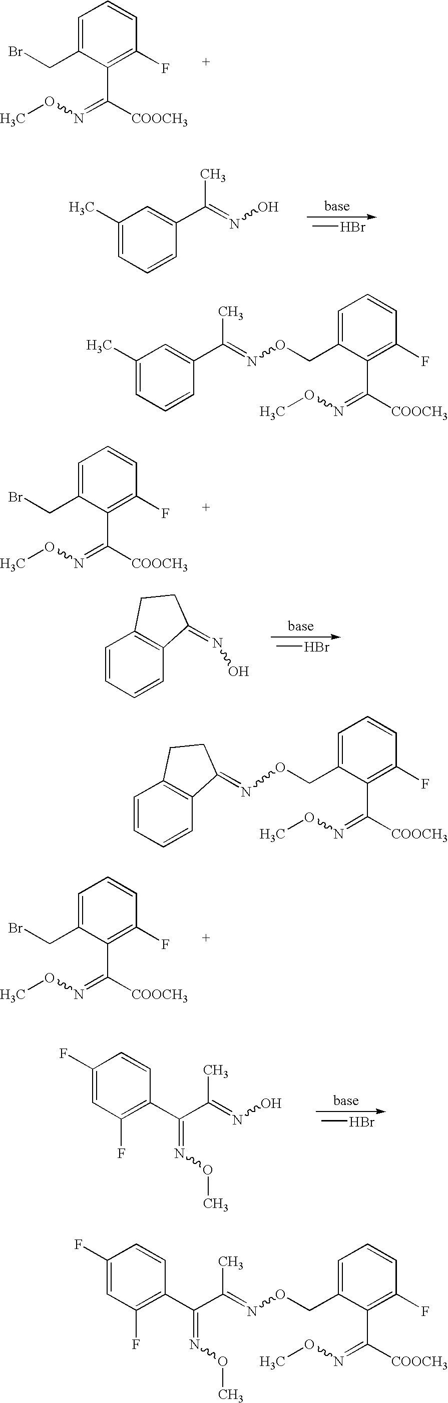

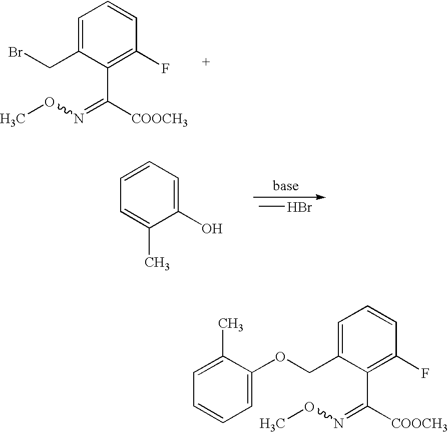

The formula (VIII) provides a general definition of the 2-(2-halomethylphenyl)-2-methoxyimino derivatives required as starting materials for carrying out the process d) according to the invention. In this formula (VIII), R², R³, R⁴ and T preferably or in particular have those meanings which have already been given in connection with the description of the compounds of the formula (I) according to the invention as being preferred or as being particularly preferred for R², R³, R⁴ and T. Y⁶ represents halogen, preferably chlorine or bromine.

The compounds of the formula (VIII) are novel and also form part of the subject-matter of the present application. They are obtained when (process m) a 2-(2-methylphenyl)-2-methoxyimino derivative of the formula (XVIII) 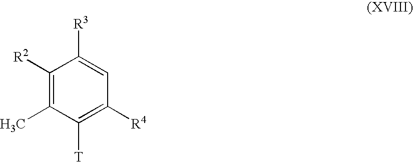
in which
R², R³, R⁴ and T are as defined above
is reacted with a halogenating agent, if appropriate in the presence of a diluent and if appropriate in the presence of a catalyst.

The process m) according to the invention can be illustrated by the following reaction equation:

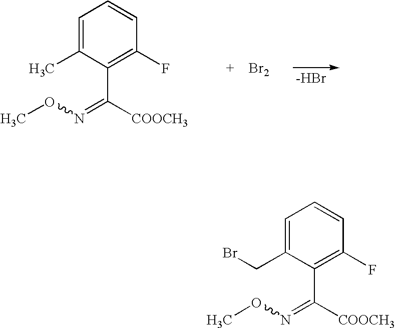

The formula (XVIII) provides a general definition of the 2-(2-methylphenyl)-2-methoxyimino derivatives required as starting materials for carrying out the process m) according to the invention. In this formula (XVIII), R², R³, R⁴ and T preferably or in particular have those meanings which have already been given in connection with the description of the compounds of the formula (I) according to the invention as being preferred or as being particularly preferred for R², R³, R⁴ and T.

The 2-(2-methylphenyl)-2-methoxyimino derivatives of the formula (XVIII) are novel and also form part of the subject-matter of the present application. They are obtained when (process n) O-tolyl pyruvates of the formula (XIX) 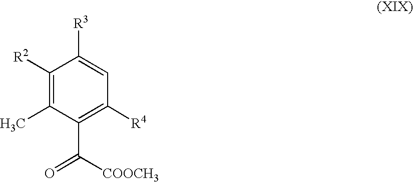
in which
R², R³ and R⁴ are as defined above
are reacted with methoxyamine or one of its salts, if appropriate in the presence of a diluent, or when (process o) a 2-(2-methylphenyl)-2-methoxyimino derivative of the formula (XVIII-a) 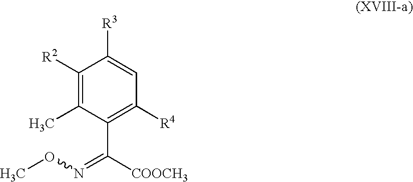
   in which
R², R³ and R⁴ are as defined above
is reacted with methylamine, if appropriate in the presence of a diluent,
or when (process q) a 2-(2-methylphenyl)-2-methoxyimimo derivative of the formula (XVIII-a) is, in a first step, reacted with hydroxylamine or a salt thereof, if appropriate in the presence of an acid acceptor and if appropriate in the presence of a diluent, and, without isolation of the product of the first step, reacted in a second step with dibromoethane, if appropriate in the presence of an acid acceptor and if appropriate in the presence of a diluent.

The process n) according to the invention can be illustrated by the following reaction equation:

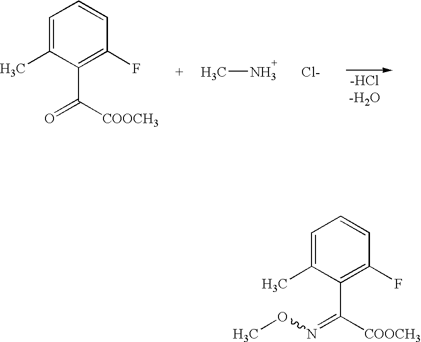

The formula (XIX) provides a general definition of the O-tolyl pyruvates required as 10 starting materials for carrying out the process n) according to the invention. In this formula (XIX), R², R³ and R⁴ preferably or in particular have those meanings which have already been given in connection with the description of the compounds of the formula (XIX) according to the invention as being preferred or as being particularly preferred for R², R³ and R⁴.

The O-tolyl pyruvates of the formula (XIX) are novel and also form part of the subject-matter of the present application. They are obtained (process p), when benzoyl cyanides of the formula (XX) 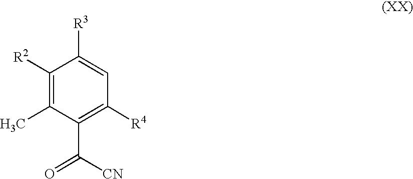
in which
R², R³ and R⁴ are as defined above are hydrolysed and esterified in the presence of methanol and, if appropriate, in the presence of an acid.

The process p) according to the invention can be illustrated by the reaction equation below:

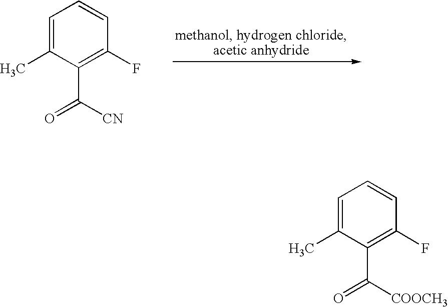

The formula (XX) provides a general definition of the benzoyl cyanides required as starting materials for carrying out the process p) according to the invention. In this formula (XX), R², R³ and R⁴ preferably or in particular have those meanings which have already been given in connection with the description of the compounds of the formula (I) according to the invention as being preferred or as being particularly preferred for R², R³ and R⁴.

The benzoyl cyanides of the formula (XX) are novel and also form part of the subject-matter of the present application.

They are obtained (process r) when benzoic acid derivatives of the formula (XXI) 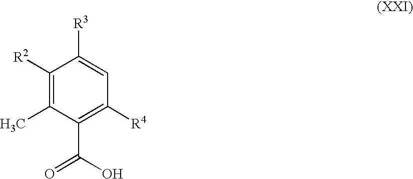
in which
R², R³ and R⁴ are as defined above
are, in a first step, reacted with a chlorinating agent, if appropriate in the presence of a catalyst and if appropriate in the presence of a diluent, and, if appropriate without isolation of the product of the first step, reacted in a second step with trimethylsilyl cyanide.

The process r) according to the invention can be illustrated by the reaction equation below:

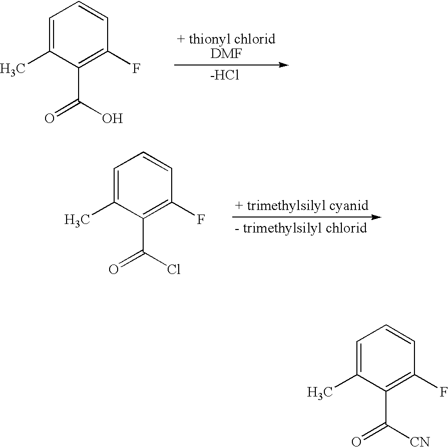

The formula (XXI) provides a general definition of the benzoic acid derivatives required as starting materials for carrying out the process r) according to the invention. In this formula (XXI), R², R³ and R⁴ preferably or in particular have those meanings which have already been given in connection with the description of the compounds of the formula (I) according to the invention as being preferred or as being particularly preferred for R², R³ and R⁴.

The benzoic acid derivatives of the formula (XXI) are known or can be prepared by known methods (compare, for example, DE 3328494).

The process o) according to the invention can be illustrated by the reaction equation below:

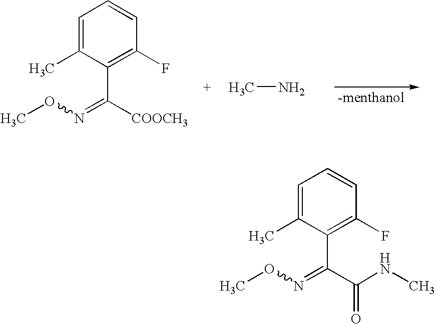

The formula (XVIII-a) provides a general definition of the 2-(2-methylphenyl)-2-methoxyimino derivatives required as starting materials for carrying out the process o) according to the invention. In this formula (XVIII-a), R², R³ and R⁴ preferably or in particular have those meanings which have already been given in connection with the description of the compounds of the formula (I) according to the invention as being preferred or as being particularly preferred for R², R³ and R⁴.

The 2-(2-methylphenyl)-2-methoxyimino derivatives of the formula (XVIII-a) are compounds according to the invention and can be prepared by process n).

Methylamine, furthermore required as starting material for carrying out the process o) according to the invention, is a commercial laboratory chemical.

The process q) according to the invention can be illustrated by the reaction equation below:

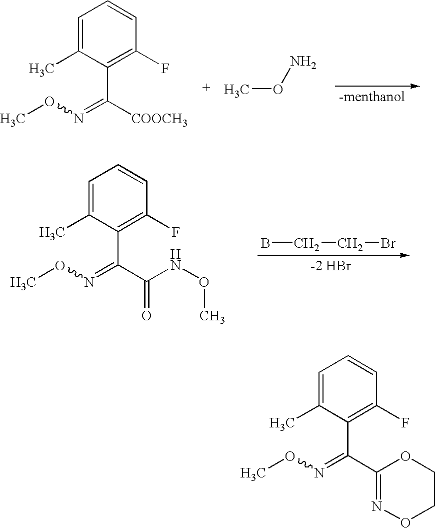

The formula (XVIII-a) provides a general definition of the 2-(2-methylphenyl)-2-methoxyimino derivatives required as starting materials for carrying out the process q) according to the invention; these compounds have already been described in the description of process n) according to the invention.

The substances hydroxylamine or its salts and dibromoethane, furthermore required as starting materials for carrying out the process q) according to the invention, are commercial laboratory chemicals.

Halogenating agents suitable for carrying out the process m) according to the invention are, preferably, all customary chlorinating, brominating and iodinating agents. The following compounds may be mentioned by way of example: chlorine, bromine, iodine, chlorosuccinimide, bromosuccinimide, 1,3-dibromo-5,5-dimethylhydantoin or iodosuccinimide.

All of the halogenating agents mentioned are customary laboratory chemicals.

The formula (IX) provides a general definition of the oximes furthermore required as starting materials for carrying out the process d) according to the invention. In this formula (IX), R⁶ preferably or in particular has that meaning which has already been given in connection with the description of the compounds of the formula (I) according to the invention as being preferred or as being particularly preferred for R⁶.

The oximes of the formula (IX) are known chemicals for synthesis or can be prepared by generally customary methods from ketones and hydroxylamines.

The formula (X) provides a general definition of the benzofuranoneoximes furthermore alternatively required as starting materials for carrying out the process d) according to the invention. In this formula (X), R⁷, R⁸, R⁹ and R¹⁰ preferably or in particular have those meanings which have already been given in connection with the description of the compounds of the formula (I) according to the invention as being preferred or as being particularly preferred for R⁷, R⁸, R⁹ and R¹⁰.

The benzofuranoneoximes of the formula (X) are known or can be prepared by known methods (compare, for example, WO 9621640).

The formula (XI) provides a general definition of the bisoximes furthermore alternatively required as starting materials for carrying out the process d) according to the invention. In this formula (XI), R¹¹ and R¹² preferably or in particular have those meanings which have already been given in connection with the description of the compounds of the formula (I) according to the invention as being preferred or as being particularly preferred for R¹¹and R¹².

The bisoximes of the formula (XI) are known or can be prepared by known methods (compare, for example, WO 97-24317).

The formula (XII) provides a general definition of the phenols furthermore alternatively required as starting materials for carrying out the process d) according to the invention. In this formula (XII), R⁵ preferably or in particular has that meaning which has already been given in connection with the description of the compounds of the formula (I) according to the invention as being preferred or as being particularly preferred for R⁵.

The phenols of the formula (XII) are generally known chemicals for synthesis.

The process e) according to the invention can be illustrated by the reaction equation below:

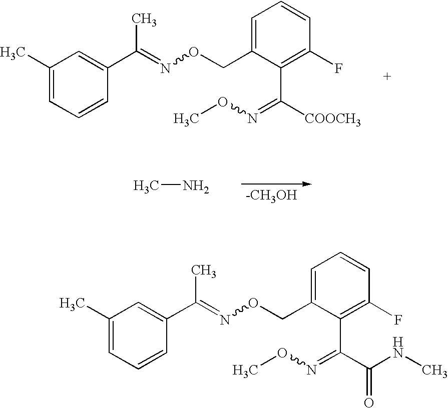

The formula (I-a) provides a general definition of the halobenzenes required as starting materials for carrying out the process e) according to the invention.

The halobenzenes of the formula (I-a) are compounds according to the invention and can be prepared by process a), b), c) or d) according to the invention.

The methylamine, furthermore required as starting material for carrying out the process e) according to the invention, is a commercial laboratory chemical.

The process f) according to the invention can be illustrated by the following reaction equation:

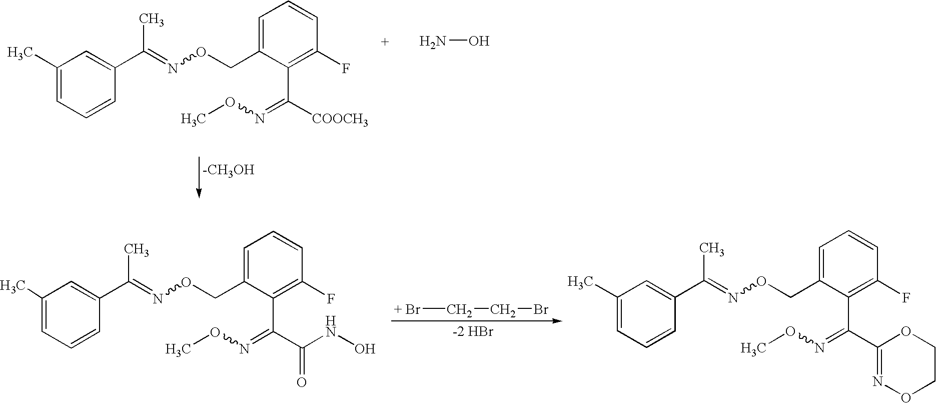

The formula (I-a) provides a general definition of the halobenzenes required as starting materials for carrying out the process f) according to the invention.

The halobenzenes of the formula (I-a) are substances according to the invention and can be prepared by process a), b), c) or d) according to the invention.

The substances hydroxylamine or its salt and dibromoethane, furthermore required as starting materials for carrying out the process f) according to the invention, are commercial laboratory chemicals.

Diluents suitable for carrying out the processes a), b) and 1) according to the invention are all inert organic solvents. These preferably include ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitrites, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; sulphoxides, such as dimethyl sulphoxide; or sulphones, such as sulpholane.

The processes a), b) and 1) according to the invention are, if appropriate, carried out in the presence of a suitable acid acceptor. Suitable acid acceptors are all customary inorganic or organic bases. These preferably include alkaline earth metal or alkali metal hydrides, hydroxides, alkoxides, carbonates or bicarbonates, such as, for example, sodium hydride, sodium amide, potassium tert-butoxide, sodium hydroxide, potassium hydroxide, sodium carbonate, potassium carbonate, potassium bicarbonate $ or sodium bicarbonate.

When carrying out the processes a), b) and 1) according to the invention, the reaction temperatures can be varied within a relatively large range. In general, the processes are carried out at temperatures of from 10° C. to 80° C., preferably at temperatures of from 20° C. to 50° C.

For carrying out the process a) according to the invention for preparing the compounds of the formula (I), in general from 1 to 5 mol, preferably from 1 to 2 mol, of the phenoxypyrimidine of the formula (III) is employed per mole of the hydroxyhalophenyl derivative of the formula (II).

For carrying out the process b) according to the invention for preparing the compounds of the formula (I), in general from 1 to 5 mol, preferably from 1 to 2 mol, of the alkoxypyrimidine of the formula (IV) are employed per mole of the hydroxyhalophenyl derivative of the formula (II).

For carrying out the process 1) according to the invention for preparing the compounds of the formula (V), in general from 1 to 15 mol, preferably from 2 to 8 mol, of the halopyrimidine of the formula (XVII) are employed per mole of the hydroxyhalophenyl derivative of the formula (II).

Diluents suitable for carrying out the process c) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

The process c) according to the invention is, if appropriate, carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium disopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process c) according to the invention, the reaction temperatures can be varied within a relatively wide range. In general, the process is carried out at temperatures of from 0° C. to 100° C., preferably at temperatures of from 20° C. to 80° C.

For carrying out the process c) according to the invention for preparing the compounds of the formula (I), in general from 1 to 2 mol, preferably from 1 to 1.5 mol, of diaryliodonium halide of the formula (VII) are employed per mole of the hydroxyhalophenyl derivative of the formula (II).

Diluents suitable for carrying out the process d) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; ketones, such as acetone, butanone, methyl isobutyl ketone or cyclohexanone; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate; sulphoxides, such as dimethyl sulphoxide; sulphones, such as sulpholane.

The process d) according to the invention is, if appropriate, carried out in the presence of a suitable acid acceptor. Suitable acid acceptors are all customary inorganic or organic bases. These preferably include alkaline earth metal or alkali metal hydrides, hydroxides, amides, alkoxides, acetates, carbonates or bicarbonates, such as, for example, sodium hydride, sodium amide, lithium diisopropylamide, sodium methoxide, sodium ethoxide, potassium tert-butoxide, sodium hydroxide, potassium hydroxide, sodium acetate, sodium carbonate, potassium carbonate, potassium bicarbonate, sodium bicarbonate or ammonium carbonate, and also tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process d) according to the invention, the reaction temperatures can be varied within a relatively wide range. In general, the process is carried out at temperatures of from 0° C. to 100° C., preferably at temperatures of from 20° C. to 80° C.

For carrying out the process d) according to the invention for preparing the compounds of the formula (I), in general from 1 to 5 mol, preferably from 1 to 2 mol, of the oxime of the formula (IX) or benzofuranoneoxime of the formula (X) or bisoxime of the formula (XI) are employed per mole of the 2-(2-halomethylphenyl)-2-methoxyimino derivative of the formula (VIII).

Suitable diluents for carrying out the processes e), h,) and o) according to the invention are all inert organic solvents. These preferably include aliphatic, alicyclic or aromatic hydrocarbons, such as, for example, petroleum ether, hexane, heptane cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitrites, such as acetonitrile, propionitrile, n- or i-butyronitrile or benzonitrile; amides, such as N,N-dimethylformamide, N,N-dimethylacetamide, N-methylformanilide, N methylpyrrolidone or hexamethylphosphoric triamide; esters, such as methyl acetate or ethyl acetate; sulphoxides, such as dimethyl sulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether.

When carrying out the processes e), h) and o) according to the invention, the reaction temperatures can be varied within a relatively wide range. In general, the processes are carried out at temperatures of from 0° C. to 100° C., preferably at temperatures of from 0° C. to 60° C.

For carrying out the process e) according to the invention for preparing the compounds of the formula (1), in general from 1 to 100 mol, preferably from 1 to 10 mol, of methylamine are employed per mole of the halobenzene of the formula (I-a).

For carrying out the process h) according to the invention for preparing the compounds of the formula (11), in general from 1 to 100 mol. preferably from 1 to 10 mol, of methylamine are employed per mole of the hydroxyhalophenyl derivative of the formula (II-a).

For carrying out the process o) according to the invention for preparing the compounds of the formula (XVI), in general from 1 to 100 mol, preferably from 1 to 10 mol, of methylamine are employed per mole of the 2-(2-methylphenyl)-2-methoxyimino derivative of the formula (XV-a).

Diluents suitable for carrying out the processes f) and q) according to the invention are polar solvents. These preferably include alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

When carrying out the processes f) and q) according to the invention, the reaction temperatures can be varied within a relatively wide range. In general, the processes are carried out at temperatures of from 0° C. to 150° C., preferably at temperatures of from 0° C. to 80° C.

For carrying out the process f) according to the invention, in general from 1 to 5 mol, preferably from 1.0 to 2.5 mol, of hydroxylamine or hydroxylamine hydrogen halide and in general from 1 to 10 mol, preferably from 1.0 to 5.0 mol, of dibromoethane are employed per mole of halobenzene of the formula (I-a).

For carrying out the process q) according to the invention, in general from 1 to 5 mol, preferably from 1.0 to 2.5 mol, of hydroxylamine or hydroxylamine hydrogen halide and in general from 1 to 10 mol, preferably from 1.0 to 5.0 mol, of dibromoethane are employed per mole of 2-(2-methylphenyl)-2-methoxyimino derivative of the formula (XVIII-a).

Diluents suitable for carrying out the processes g) and n) according to the invention are polar solvents. These preferably include alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

When carrying out the processes g) and n) according to the invention, the reaction temperatures can be varied within a relatively wide range. In general, the processes are carried out at temperatures of from 0° C. to 150° C., preferably at temperatures of from 0° C. to 80° C.

For carrying out the process g) according to the invention for preparing the compounds of the formula (II), in general from 1 to 15 mol, preferably from 1 to 8 mol, of methoxyamine hydrochloride are employed per mole of the ketoester of the formula (XIII) or the hydroxyphenylglyoxylic acid esters of the formula (XIV).

For carrying out the process n) according to the invention for preparing the compounds of the formula (XVIII), in general from 1 to 15 mol, preferably from 1 to 8 mol, of methoxyamine or one of its salts are employed per mole of the O-tolyl pyruvate of the formula (XIX).

Diluents suitable for carrying out the process i) according to the invention are inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole.

The process i) according to the invention is, if appropriate, carried out in the presence of a strong base. These preferably include n-, s-, t-butylithium or lithium diisopropylamide.

When carrying out the process i) according to the invention, the reaction temperatures can be varied within a relatively wide range. In general, the process is carried out at temperatures of from −150° C. to 0° C., preferably at temperatures of from −100° C. to −40° C. For carrying out the process i) according to the invention for preparing the compounds of the formula (XIII), in general from 1 to 5 mol, preferably from 1 to 2 mol, of dimethyl oxalate are employed per mole of the ketal of the formula (XV).

Diluents suitable for carrying out the process j) according to the invention are inert organic solvents. These preferably include aliphatic or alicyclic hydrocarbons, such as, for example, petroleum ether, hexane, heptane, cyclohexane, methylcyclohexane or decalin; halogenated hydrocarbons, such as, for example, chlorobenzene, dichlorobenzene, dichloromethane, chloroform, carbon tetrachloride, dichloroethane or trichloroethane.

The process j) according to the invention is, if appropriate, carried out in the presence of a Lewis acid. These include, for example, boron trifluoride (also as etherate), boron tribromide, aluminium trichloride, titanium tetrachloride, tetrabutylorthotitanate, zinc chloride, iron(III) chloride or antimony pentachloride.

When carrying out the process j) according to the invention, the reaction temperatures can be varied within a relatively wide range. In general, the process is carried out at temperatures of from 0° C. to 150° C., preferably at temperatures of from 0° C. to 80° C.

For carrying out the process j) according to the invention for preparing the compounds of the formula (XIV), in general from 1 to 5 mol, preferably from 1 to 2 mol, of methyl oxalyl chloride are employed per mole of the fluorophenol of the formula (XVI).

Diluents suitable for carrying out the process k) according to the invention are inert organic solvents. These preferably include ethers, such as diethyl ether, diisopropyl ether, methyl t-butyl ether, methyl t-amyl ether, diloxane, tetrahydrofuran, 1,2-dimethoxyethane, 1,2-diethoxyethan or anisole; alcohols, such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water or pure water.

The process k) according to the invention is, if appropriate, carried out in the presence of an acid. Suitable acids are all inorganic and organic protic acids and also Lewis acids, as well as polymeric acids. These include, for example, hydrogen chloride, sulphuric acid, phosphoric acid, formic acid, acetic acid, trifluoroacetic acid, methanesulphonic acid, trifluoromethanesulphonic acid, toluenesulphonic acid, boron trifluoride (also as etherate), boron tribromide, aluminium trichloride, titanium tetrachloride, tetrabutylorthotitanate, zinc chloride, iron(III) chloride, antimony pentachloride, acidic ion exchangers, acidic clays and acidic silica gels.

When carrying out the process k) according to the invention, the reaction temperatures can be varied within a relatively wide range. In general, the process is carried out at temperatures of from 0° C. to 120° C., preferably at temperatures of from 0° C. to 50° C.

Diluents suitable for carrying out the process m) according to the invention are inert organic solvents. These preferably include halogenated hydrocarbons, such as, for example, chlorobenzene, dichlorobenzene, dichloromethane, chloroform, carbon tetrachloride, dichloroethane or trichloroethane.

When carrying out the process m) according to the invention, the reaction temperatures can be varied within a relatively wide range. In general, the process is carried out at temperatures of from 0° C. to 150° C., preferably at temperatures of from 0° C. to 100° C.

For carrying out the process m) according to the invention for preparing the compounds of the formula (VIII), in general from 1 to 1.5 mol, preferably from 1 to 1.2 mol, of halogenating agent are employed per mole of the 2-(2-methylphenyl)-2-methoxyimino derivative of the formula (XVIII).

Diluents suitable for carrying out the process p) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole.

When carrying out the process p) according to the invention, the reaction temperatures can be varied within a relatively wide range. In general, the process is carried out at temperatures of from −20° C. to 80° C., preferably at temperatures of from 0° C. to 50° C.

For carrying out the process p) according to the invention for preparing the compounds of the formula (XIX), in general from 1 to 5 mol, preferably from 1 to 2 mol, of acetic anhydride are employed per mole of the benzoyl cyanide of the formula (XX).

Diluents suitable for carrying out the first step of the process r) according to the invention are all inert organic solvents. These preferably include aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as, for example, chlorobenzene, dichlorobenzene, dichloromethane, chloroform, carbon tetrachloride, dichloroethane or trichloroethane.

Chlorinating agents suitable for carrying out the first step of the process r) according to the invention are all customary chlorinating agents which can be used for preparing acid chlorides, such as, for example, thionyl chloride, phosphorus trichloride or phosphorus pentachloride.

The process r) according to the invention is, if appropriate, carried out in the presence of a catalyst, such as, for example, dimethylformamide or dimethylacetamide.

When carrying out both the first and the second step of the process r) according to the invention, the reaction temperatures can be varied within a relatively wide range. In general, the first step is carried out at temperatures of from 20° C. to 150° C., preferably at temperatures of from 20° C. to 100° C., and the second step is carried out at temperatures of from 100° C. to 250° C., preferably at temperatures of from 100° C. to 200° C.

For carrying out the process r) according to the invention for preparing the compounds of the formula (XX), in general from 1 to 15 mol, preferably from 1 to 8 mol, of halogenating agent and from 1 to 5 mol, preferably from 1 to 2 mol, of trimethylsilyl cyanide are employed per mole of the benzoic acid derivative of the formula (XXI).

All of the processes according to the invention are generally carried out under atmospheric pressure. However, it is also possible to operate under elevated or reduced pressure—in general between 0.1 bar and 10 bar.

The reaction, work-up and isolation of the reaction products are carried out by generally customary methods (compare also the Preparation Examples).

The active compounds are suitable for controlling animal pests, in particular insects, arachnids and nematodes, which are encountered in agriculture, in forests, in the protection of stored products and of materials, and in the hygiene sector because they are tolerated well by plants and have favourable homeotherm toxicity. They may preferably be employed as crop protection agents. They are active against normally sensitive and resistant species and against all or some stages of development. The abovementioned pests include:
From the order of the Isopoda, for example, Oniscus asellus, Armadillidium vulgare and Porcellio scaber.
From the order of the Diplopoda, for example, Blaniulus guttulatus.
From the order of the Chilopoda, for example, Geophilus carpophagus and Scutigera spp.
From the order of the Symphyla, for example, Scutigerella immaculata.
From the order of the Thysanura, for example, Lepisma saccharina.
From the order of the Collembola, for example, Onychiurus armatus.
From the order of the Orthoptera, for example, Acheta domesticus, Gryllotalpa spp., Locusta migratoria migratorioides, Melanoplus spp. and. Schistocerca gregaria.
From the order of the Blattaria, for example, Blatta orientalis, Periplaneta americana, Leucophaea maderae and Blattella germanica.
From the order of the Dermaptera, for example, Forficula auricularia.
From the order of the Isoptera, for example, Reticulitermes spp.
From the order of the Phthiraptera, for example, Pediculus humanus corporis, Haematopinus spp., Linognathus spp., Trichodectes spp. and Damalinia spp.
From the order of the Thysanoptera, for example, Hercinothrips femoralis, Thrips tabaci, Thrips palmi and Frankliniella occidentalis.
From the order of the Heteroptera, for example, Eurygaster spp., Dysdercus intermedius, Piesma quadrata, Cimex lectularius, Rhodnius prolixus and Triatoma spp.
From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyaloperus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium comi, Saissetia oleae, Laodelphax striatellus, Nilaparvata lugens, Aonidiella aurantii, Aspidiotus hederae, Pseudococcus spp. and Psylla spp.
From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura furniferana, Clysia ambiguella, Homona magnanima, Tortrix viridana, Cnaphalocerus spp. and Oulema oryzae.
From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.
From the order of the Hymenoptera, for example, Diprion spp., Hoplocampa spp., Lasius spp., Monomoriun pharaonis and Vespa spp.
From the order of the Diptera, for example, Aedes spp., Anopheles spp., Culex spp., Drosophila melanogaster, Musca spp., Fannia spp., Calliphora erytlirocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratifis capitata, Dacus oleae, Tipula paludosa, Hylemyia spp. and Liriomyza spp.
From the order of the Siphonaptera, for example, Xenopsylla cheopis and Ceratophyllus spp.
From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

They can be empolyed with particularly good results for controlling plant-damaging insects, such as, for example, against the larvae of the green peach aphid (Myzus persicae) and the caterpillars of the green army worm (Spodoptera frugiperda).

The substances according to the invention have potent microbicidal activity and can be employed for controlling undesirable microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases Which come under the generic names listed above may be mentioned as examples, but not by way of limitation:
Xanthomonas species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also have very good fortifying action in plants. Accordingly, they can be used for mobilizing the defences of the plant against attack by undesirable microorganisms.

In the present context, plant-fortifying (resistance-inducing) substances are to be understood as meaning those substances which are capable of stimulating the defence system of plants such that, when the treated plants are subsequently inoculated with undesirable microorganisms, they show substantial resistance to these microorganisms.

In the present case, undesirable microorganisms are to be understood as meaning phytopathogenic fungi, bacteria and viruses. Accordingly, the substances according to the invention can be used to protect plants for a certain period after the treatment against attack by the pathogens mentioned. The period for which protection is provided generally extends over 1 to 10 days, preferably 1 to 7 days, after the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be used with particularly good results for controlling cereal diseases, such as, for example, against Erysiphe or Leptosphaeria species, of diseases in viticulture and in the cultivation of fruit and vegetables, such as, for example, against Venturia, Sphaerotheca, Podosphaera and Phytophtora species.

The active compounds according to the invention are also suitable for increasing the yield of crops. In addition, they show reduced toxicity and are well tolerated by plants.

At certain concentrations and application rates, the active compounds according to the invention can also be used as herbicides, for influencing plant growth and for controlling animal pests. If appropriate, they can also be used as intermediates and precursors for the synthesis of further active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on, and, in the case of propagation material, in particular in the case of seeds, also by applying one or more coats.

In the protection of materials, the substances according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be adhesives, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular moulds, wood-discolouring and wood-destroying fungi (Basidiomycetes), and against slime organisms and algae.

Microorganisms of the following genera may be mentioned as examples:
Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted to the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils. It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can be used as such or in their formulations, also in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Examples of suitable mixing components are the following compounds

Fungicides:

aldimorph, ampropylfos, ampropylfos potassium, andoprim, anilazine, azaconazole, azoxystrobin,
benalaxyl, benodanil, benomyl, benzamacril, benzamacril-isobutyl, bialaphos, binapacryl, biphenyl, bitertanol, blasticidin-S, bromuconazole, bupirimate, buthiobate,
calcium polysulphide, carpropamid, capsimycin, captafol, captan, carbendazim, carboxin, carvon, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram,
debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,
edifenphos, epoxiconazole, etaconazole, ethirimol, etridiazole,
famoxadon, fenapanil, fenarimol, fenbuconazole, fenfuram, fenhexamid, fenitropan, fenpiclonil, fenpropidin, fenpropimorph, fentin acetate, fentin hydroxide, ferbarm, ferimzone, fluazinam, flumetover, fluoromide, fluquinconazole, flurprimidol, flusilazole, flusulfamide, flutolanil, flutriafol, folpet, fosetyl-aluminium, fosetyl-sodium, fthalide, fuberidazole, furalaxyl, furametpyr, furcarbonil, furconazole, furconazole-cis, furmecyclox,
guazatine,
hexachlorobenzene, hexaconazole, hymexazole,
imazalil, imibenconazole, iminoctadine, iminoctadine albesilate, iminoctadine triacetate, iodocarb, ipconazole, iprobenfos (IBP), iprodione, iprovalicarb irumamycin, isoprothiolane, isovaledione,
kasugamycin, kresoxim-methyl, copper preparations, such as: copper hydroxide, copper naphthenate, copper oxychloride, copper sulphate, copper oxide, oxine-copper and Bordeaux mixture,
mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,
nickel dimethyldithiocarbamate, nitrothal-isopropyl, nuarimol,
ofurace, oxadixyl, oxamocarb, oxolinic acid, oxycarboxim, oxyfenthin,
paclobutrazole, pefurazoate, penconazole, pencycuron, phosdiphen, picoxystrobin, pimaricin, piperalin, polyoxin, polyoxorim, probenazole, prochloraz, procymidone, propamocarb, propanosine-sodium, propiconazole, propineb, pyraclostrobin, pyrazophos, pyrifenox, pyrimethanil, pyroquilon, pyroxyfur,
quinconazole, quintozene (PCNB), quinoxyfen,
sulphur and sulphur preparations, spiroxamines,
tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamide, tricyclazole, tridemorph, trifloxystrobin, triflumizole, triforine, triticonazole, uniconazole,
validamycin A, vinclozolin, viniconazole,
zarilamide, zineb, ziram and also
Dagger G,
OK-8705,
OK-8801,
α-(1,1-dimethylethyl)-β-(2-phenoxyethyl)-1H-1,2,4-trialzole-1-ethanol,
α-(2,4-dichlorophenyl)-β-fluoro-β-propyl-1H-1,2,4-triazole-1-ethanol,
α-(2,4-dichlorophenyl)-β-methoxy-α-methyl-1H-1,2,4-triazole-b 1-ethanol,
α-(5-methyl-1,3-dioxan-5-yl)-β-[[4-(trifluoromethyl)-phenyl]-methylene]-1H-1,2,4-triazole-1-ethanol,
(5RS,6RS)-6-hydroxy-2,2,7,7-tetramethyl-5-(1H-1,2,4-triazol-1-yl)-3-octanone,
(E)-α-(methoxyimino)-N-methyl-2-phenoxy-phenylacetamide,
1-(2,4-dichlorophenyl)-2-(1H-1,2,4-triazol-1-yl)-ethanone O-(phenylmethyl)-oxime,
1-(2-methyl-1-naphthalenyl)-1H-pyrrole-2,5-dione,
1-(3,5-dichlorophenyl)-3-(2-propenyl)-2,5-pyrrolidinedione,
1-[(diiodomethyl)-sulphonyl]-4-methyl-benzene,
1-[[2-(2,4-dichlorophenyl)-1,3-dioxolan-2-yl]-methyl]-1H-imidazole,
1-[[2-(4-chlorophenyl)-3-phenyloxiranyl]-methyl]-1H-1,2,4-triazole,
1-[1-[2-[(2,4-dichlorophenyl)-methoxy]-phenyl]-ethenyl]-1H-imidazole,
1-methyl-5-nonyl-2-(phenylmethyl)-3-pyrrolidinole,
2′,6′-dibromo-2-methyl-4′-trifluoromethoxy-4′-trifluoromethyl-1,3-thiazole-5-carboxanilide,
2,6-dichloro-5-(methylthio)-4-pyrimidinyl-thiocyanate,
2,6-dichloro-N-(4-trifluoromethylbenzyl)-benzamide,
2,6-dichloro-N-[[4-(trifluoromethyl)-phenyl]-methyl]-benzamide,
2-(2,3,3-triiodo-2-propenyl)-2H-tetrazole,
2-[(1-methylethyl)-sulphonyl]-5-(trichloromethyl)-1,3,4-thiadiazole,
2-[[6-deoxy-4-O-(4-O-methyl-β-D-glycopyranosyl)-α-D-glucopyranosyl]-amino]-4-methoxy-1H-pyrrolo[2,3-d]pyrimidine-5-carbonitrile,
2-aminobutane,
2-bromo-2-(bromomethyl)-pentanedinitrile,
2-chloro-N-(2,3-dihydro-1,1,3-trimethyl-1H-inden-4-yl)-3-pyridinecarboxamide,
2-chloro-N-(2,6-dimethylphenyl)-N-(isothiocyanatomethyl)-acetamiide,
2-phenylphenol (OPP),
3,4-dichloro-1-[4-(difluoromethoxy)-phenyl]-1H-pyrrole-2,5-dione,
3,5-dichloro-N-[cyano[(1-methyl-2-propynyl)-oxy]-methyl]-benzamide,
3-(1,1-dimethylpropyl-1-oxo-1H-indene-2-carbonitrile,
3-[2-(4-chlorophenyl)-5-ethoxy-3-isoxazolidinyl]-pyridine,
4-chloro-2-cyano-N,N-dimethyl-5-(4-methylphenyl)-1H-imidazole-1-sulphonamide,
4-methyl-tetrazolo[1,5-a]quinazolin-5(4H)-one,
8-hydroxyquinoline sulphate,
9H-xanthene-2-[(phenylamino)-carbonyl]-9-carboxylic hydrazide,
bis-(1-methylethyl)-3-methyl-4-[(3-methylbenzoyl)-oxy]-2,5-thiophenedicarboxylate,
cis-1-(4-chlorophenyl)-2-(1H-1,2,4-triazol-1-yl)-cycloheptanol,
cis-4-[3-[4-(1,1-dimethylpropyl)-phenyl-2-methylpropyl]-2,6-dimethyl-morpholine-hydrochloride,
ethyl [(4-chlorophenyl)-azo]-cyanoacetate,
potassium bicarbonate,
methanetetrathiol sodium salt,
methyl 1-(2,3-dihydro-2,2-dimethyl-1H-inden-1-yl)-1H-imidazole-5-carboxylate,
methyl N-(2,6-dimethylphenyl)-N-(5-isoxazolylcarbonyl)-DL-alaninate,
methyl N-(chloroacetyl)-N-(2,6-dimethylphenyl)-DL-alaninate,
N-(2,6-dimethylphenyl)-2-methoxy-N-(tetrahydro-2-oxo 3-furanyl)-acetamide,
N-(2,6-dimethylphenyl)-2-methoxy-N-(tetrahydro-2-oxo-3-thienyl)-acetamide,
N-(2-chloro-4-nitrophenyl)-4-methyl-3-nitro-benzenesulphonamide,
N-(4-cyclohexylphenyl)-1,4,5,6-tetrahydro-2-pyrimidineamine,
N-(4-hexylphenyl)-1,4,5,6-tetrahydro-2-pyrimidineamine,
N-(5-chloro-2-methylphenyl)-2-methoxy-N-(2-oxo-3-oxazolidinyl)-acetamide,
N-(6-methoxy-3-pyridinyl)-cyclopropanecarboxamide,
N-[2,2,2-trichloro-1-[(chloroacetyl)-amino]-ethyl]-benzamide,
N-[3-chloro-4,5-bis-(2-propinyloxy)-phenyl]-N′-methoxy-methanimidamide,
N-formyl-N-hydroxy-DL-alanine-sodium salt,
O,O-diethyl [2-(dipropylamino)-2-oxoethyl]-ethylphosphoramidothioate,
O-methyl S-phenyl phenylpropylphosphoramidothioate,
S-methyl 1,2,3-benzothiadiazole-7-carbothioate,
spiro[2H]-1-benzopyrane-2,1′ (3′H)-isobenzofuran]-3′-one,
4-[3,4-dimethoxyphenyl)-3-(4-fluorophenyl)-acryloyl]-morpholine
   Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.
   Insecticides/acaricides/nematicides:
abamectin, acephate, acetamiprid, acrinathrin, alanycarb, aldicarb, aldoxycarb, alpha-cypermethrin, alphamethrin, amitraz, avermectin, AZ 60541, azadirachtin, azamethiphos, azinphos A, azinphos M, azocyclotin,
Bacillus popilliae, Bacillus sphaericus, Bacillus subtilis, Bacillus thuringiensis, baculoviruses, Beauveria bassiana, Beauveria tenella , bendiocarb, benfiiracarb, bensultap, benzoximate, betacyfluthrin, bifenazate, bifenthrin, bioethanomethrin, biopermetrin, bistrifluron, BPMC, bromophos A, bufencarb, buprofezin, butathiofos, butocarboxim, butylpyridaben,
cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, chromafenozide, cis-resmethrin, cispermethrin, clocythrin, cloethocarb, clofentezine, clothianidine, cyanophos, cycloprene, cycloprothrin, cyfluthrin, cyhalothrin, cyhexatin, cypermethrin, cyromazme,
deltamethrin, demeton M, demeton S, demeton-S-methyl, diafenthiuron, diazinon, dichlorvos, dicofol, diflubenzuron, dimethoat, dimethylvinphos, diofenolan, disulfoton, docusat-sodium, dofenapyn,
eflusilanate, emamectin, empenthrin, endosulfan, Entomopfthora spp., esfenvalerate, ethiofencarb, ethion, ethoprophos, etofenprox, etoxazole, etrimfos,
fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flumethrin, flutenzine, fluvalinate, fonophos, fosmethilan, fosthiazate, fabfenprox, furathiocarb,
granulosis viruses,
halofenozide, HICH, heptenophos, hexaflumuron, hexythiazox, hydroprene,
imidacloprid, indoxacarb, isazofos, isofenphos, isoxathion, ivermectin,
nuclear polyhedrosis viruses,
lambda-cyhalothrin, lufenuron,
malathion, mecarbam, metaldehyde, methamidophos, Metharhizium anisopliae, Metharhizium flavoviride, methidathion, methiocarb, methoprene, methomyl, methoxyfenozide, metolcarb, metoxadiazone, mevinphos, milbemectin, milbemycin, monocrotophos,
naled, nitenpyram, nithiazine, novaluron,
omethoate, oxamyl, oxydemethon M,
Paecilomyces fumosoroseus, parathion A, parathion M, permethrin, phenthoate, phorat, phosalone, phosmet, phosphamidon, phoxim, pirimicarb, pirimiphos A, pirimiphos M, profenofos, promecarb, propargite, propoxur, prothiofos, prothoat, pymetrozine, pyraclofos, pyresmethrin, pyrethrum, pyridaben, pyridathion, pyrimidifen, pyriproxyfen,
quinalphos,
ribavirin,
salithion, sebufos, silafluofen, spinosad, spirodiclofen, sulfotep, sulprofos,
tau-fluvalinate, tebufenozide, tebufenpyrad, tebupirimiphos, teflubenzuron, tefluthrin, temephos, temivinphos, terbufos, tetrachlorvinphos, tetradifon theta-cypermethrin, thiacloprid, thiamethoxam, thiapronil, thiatriphos, thiocyclam hydrogen oxalate, thiodicarb, thiofanox, thuringiensin, tralocythrin, tralomethrin, triarathene, triazamate, triazophos, triazuron, trichlophenidine, trichlorfon, triflumuron, trimethacarb,
vamidothion, vaniliprole, Verticillium lecanii,
YI 5302
zeta-cypermethrin, zolaprofos
(1R-cis)-[5-(phenylmethyl)-3-furanyl]-methyl 3-[(dihydro-2-oxo-3(2H)-furanylidene)-methyl]-2,2-dimethylcyclopropanecarboxylate
(3-phenoxyphenyl)-methyl 2,2,3,3-tetramethylcyclopropanecarboxylate,
1-[(2-chloro-5-thiazolyl)methyl]tetrahydro-3,5-dimethyl-N-nitro-1,3,5-triazine-2(1H)-imine,
2-(2-chloro-6-fluorophenyl)-4-[4-(1,1-dimethylethyl)phenyl]-4,5-dihydro-oxazole,
2-(acetyloxy)-3-dodecyl-1,4-naphthalenedione,
2-chloro-N-[[[4-(1-phenylethoxy)-phenyl]-amino]-carbonyl]-benzamide,
2-chloro-N-[[[4-(2,2-dichloro-1,1-difluoroethoxy)-phenyl]-amino]-carbonyl]-benzamide,
3-methylphenyl propylcarbamate
4-[4-(4-ethoxyphenyl)-4-methylpentyl]-1-fluoro-2-phenoxy-benzene,
4-chloro-2-(1,1-dimethylethyl)-5-[[2-(2,6-dimethyl-4-phenoxyphenoxy)ethyl]thio]-3(2H)-pyridazinone,
4-chloro-2-(2-chloro-2-methylpropyl)-5-[(6-iodo-3-pyridinyl)methoxy]-3(2H)-pyridazinone,
4-chloro-5-[(6-chloro-3-pyridinyl)methoxy]-2-(3,4-dichlorophenyl)-3(2H)-pyridazinone,
Bacillus thuringiensis strain EG-2348,
[2-benzoyl-1-(1,1-dimethylethyl)-hydrazinobenzoic acid,
2,2-dimethyl-3-(2,4-dichlorophenyl)-2-oxo-1-oxaspiro[4.5]dec-3-en-4-yl butanoate,
[3-[(6-chloro-3-pyridinyl)methyl]-2-thiazolidinylidene]-cyanamide,
dihydro-2-(nitromethylene)-2H-1,3-thiazine-3(4H)-carboxaldehyde,
ethyl [2-[[1,6-dihydro-6-oxo-1-(phenylmethyl)-4-pyridazinyl]oxy]ethyl]-carbamate,
N-(3,4,4-trifluoro-1-oxo-3-butenyl)-glycine,
N-(4-chlorophenyl)-3-[4-(difluoromethoxy)phenyl]-4,5-dihydro-4-phenyl-1H-pyrazole-1-carboxamide,
N-[(2-chloro-5-thiazolyl)methyl]-N′-methyl-N′′-nitro-guanidine,
N-methyl-N′-(1-methyl-2-propenyl)-1,2-hydrazinedicarbothioamide,
N-methyl-N′-2-propenyl-1,2-hydrazinedicarbothioamide,
O,O-diethyl [2-(dipropylamino)-2-oxoethyl]-ethylphosphoramidothioate,
N-cyanomethyl-4-trifluoromethyl-nicotinamide,
3,5-dichloro-1-(3,3-dichloro-2-propenyloxy)-4-[3-(5-trifluoromethylpyridine-2-yloxy)-propoxy]-benzene.

A mixture with other known active compounds, such as herbicides, or with fertilizers and growth regulators, is also possible.

In addition, the compounds of the formula (I) according to the invention also have very good antimycotic activity. They have a very broad antimycotic activity spectrum in particular against dermatophytes and yeasts, moulds and diphasic fungi, (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The list of these fungi by no means limits the mycotic spectrum covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, scattering, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5000 g/ha.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as by a good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetical engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetical engineering) which are preferably treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of the plants to fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylurea, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula (I) and the mixtures of active compounds according to the invention. The preferred ranges stated above for the active compounds or mixtures also apply to the treatment of these plants. Particular emphasis is given to the treatment of plants with the compounds or mixtures specifically mentioned in the present text.

The invention is illustrated by the examples below. However, the invention is not limited to the examples.

### PREPARATION EXAMPLES

### Example 1

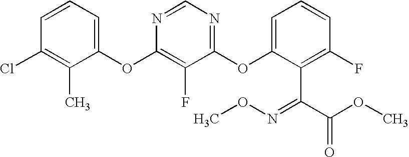 Process a)

A mixture of 1.5 g (6.6 mmol) of methyl (2-fluoro-6-hydroxyphenyl)methoxyiminoacetate, 1.7 g (6.6 mmol) of 4,5-difluoro-6-(2-methyl-3-chlorophenoxy)pyrimidine, 1.4 g (10 mmol) of potassium carbonate and 50 ml of acetonitrile is stirred at room temperature for 18 hours. The reaction mixture is then filtered through Celite, and the filtrate is concentrated under reduced pressure. This gives 2.8 g (91.5% of theory) of methyl {2-[6-(3-chloro-2-methylphenoxy)-5-fluoropyrimidin-4-yloxy]-6-fluorophenyl}methoxyiminoacetate of log P (pH 2)=4.23.

### Example 2

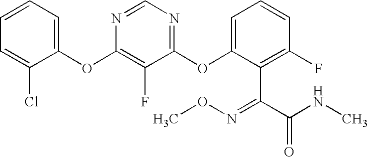 Process a)

A mixture of 0.7 g (3.1 mmol) 2-(2-fluoro-6-hydroxyphenyl)-2-methoxyimino-N-methylacetamide, 0.75 g (3.1 mmol) of 4,5-difluoro-6-(2-chlorophenoxy)pyrimidine, 0.64 g (4.6 mmol) of potassium carbonate and 50 ml of acetonitrile is stirred at room temperature for 18 hours. The reaction mixture is then filtered through Celite, and the filtrate is concentrated under reduced pressure. The residue is triturated with diisopropyl ether, and the crystalline product is filtered off with suction. This gives 0.9 g (65% of theory) of 2-{2-[6-(2-chlorophenoxy)-5-fluoropyrimidin-4-yloxy]-6-fluorophenyl}-2-methoxyimino-N-methylacetamide of log P (pH 2)=3.10.

### Example 3

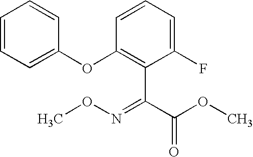 Process c)

A mixture of 1.8 g (8 mmol) of methyl (2-fluoro-6-hydroxyphenyl)methoxyiminoacetate, 2.5 g (8 mmol) of diphenyliodonium chloride, 1.7 g (12 mmol) of potassium carbonate and 60 ml of acetonitrile is boiled under reflux for 4 hours. The reaction mixture is then filtered through Celite, and the filtrate is concentrated under reduced pressure. The residue is purified by column chromatography (methylene chloride/hexane 5:1). This gives 2.04 g (84% of theory) of methyl (2-fluoro-6-phenoxyphenyl)methoxyiminoacetate of log P (pH 2)=3.24.

### Example 4

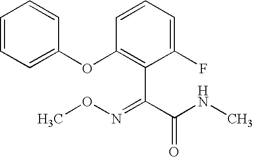 Process e)

A mixture of 2.2 g (7.25 mmol) of methyl 2-(2-fluoro-6-phenoxy)phenyl-2-methoximinoacetate, 6.2 ml of a solution of methylamino in water (40%) and 80 ml of methanol is stirred at room temperature overnight. The solvent is then distilled off under reduced pressure. This gives 1.6 g (73% of theory) of 2-(2-fluoro-6-phenoxyphenyl)-2-methoxyimino-N-methylacetamide of log P (pH 2)=2.56.

### Example 5

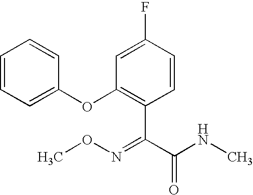 Process c)

A mixture of 1.14 g (5 mmol) of 2-(4-fluoro-2-hydroxyphenyl)-2-methoxyimino-N-methylacetamide, 1.6 g (5 mmol) of diphenyliodonium chloride, 1.05 g (7.5 mmol) of potassium carbonate and 40 ml of acetonitrile is boiled under reflux for 4 hours. The reaction mixture is then filtered through Celite, and the filtrate is concentrated under reduced pressure. The residue is triturated with diisopropyl ether, and the product is, after crystallization, filtered off with suction. This gives 1.0 g (66% of theory) of 2-(2-fluoro-6-phenoxyphenyl)-2-methoxyimino-N-methylacetamide of log P (pH 2)=2.43.

### Example 6

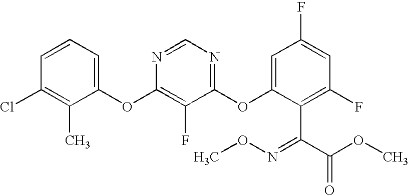 Process a)

A mixture of 0.7 g (3.1 mmol) of methyl 2-(2-hydroxy-4-fluorophenyl)-2-methoximinoacetate, 0.8 g (3.1 mmol) of 4,5-difluoro-6-(2-methyl-3-chlorophenoxy)pyrimidine, 0.64 g (4.64 mmol) of potassium carbonate and 50 ml of acetonitrile is stirred at room temperature for 18 hours. The reaction mixture is then filtered through Celite, and the filtrate is concentrated under reduced pressure. The residue is purified by column chromatography (methylene chloride). This gives 1.2 g (83.5% of theory) of methyl {2-[6-(3-chloro-2-methylphenoxy)-5-fluoropyrimidin-4-yloxy]-4-fluorophenyl}methoxyiminoacetate of log P (pH 2)=4.73.

### Example 7

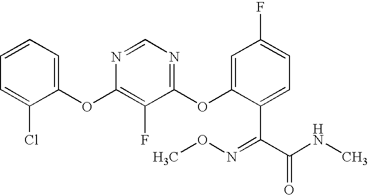 Process a)

A mixture of 0.7 g (3.1 mmol) of N-methyl-2-(2-hydroxy-4-fluorophenyl)-2-methoximinoacetamide, 0.75 g (3.1 mmol) of 4,5-difluoro-6-(2-chlorophenoxy)pyrimidine, 0.64 g (4.6 mmol) of potassium carbonate and 50 ml of acetonitrile is stirred at room temperature for 18 hours. The reaction mixture is then filtered through Celite, and the filtrate is concentrated under reduced pressure. This gives 1.2 g (87% of theory) of N-methyl-2-{2-[6-(2-chlorophenoxy)-5-fluoropyrimidin-4-yloxy]-4-fluorophenyl}-2-methoxyiminoacetamide of log P (pH 2) =3.14.

### Example 8

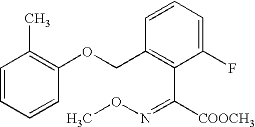 Process d)

1.12 g (0.01 mol) o-kresol are added dropwise to a suspension of 0.5 g (0.012 mol) of sodium hydride (60% in oil) in 70 ml of dimethylformamide. The mixture is stirred at room temperature for 3 hours, a solution of 4.5 g (0.01 mol) of methyl (2-bromomethyl-6-fluorophenyl)methoxyiminoacetate in 20 ml of dimethylformamide is then added and the mixture is stirred at room temperature overnight. The reaction mixture is then poured into water and extracted twice with ethyl acetate. The organic phases are washed with water, dried over sodium sulphate and concentrated under reduced pressure. The residue is chromatographed on silica gel (methylene chloride/ethyl acetate 2:1). This gives 1.6 g (48%) of methyl (2-fluoro-6-o-tolyloxymethylphenyl)methoxyiminoacetate of m.p. 108° C. and logP (pH 2)=3.65.

### Example 9

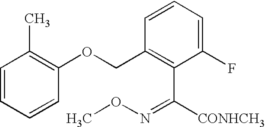 Process e)

A mixture of 0.9 g (2.7 mmol) of methyl (2-fluoro-6-o-tolyloxymethylphenyl)methoxyiminoacetate, 3 ml of a solution of methylamine in water (40%) and 30 ml of methanol is stirred at room temperature overnight. The solvent is then distilled off under reduced pressure, the residue is triturated with water and dilute hydrochloric acid and the precipitated product is filtered off with suction. This gives 0.9 g (quant.) of N-methyl-2-(2-fluoro-6-o-tolyloxymethylphenyl)-2-methoxyiminoacetamide in the form of colourless crystals of melting point 93° C. and log P (pH 2)=3.03.

The compounds of the formula (I) listed in Table 1: below are also obtained analogously to Examples 1 to 9 and in accordance with the statements in the general descriptions of processes a)-f).

TABLE 1

| | |
|---|---|
| | (I) |
| 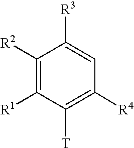 | |

| Ex. No. | | logP |
|---|---|---|
| 10 | 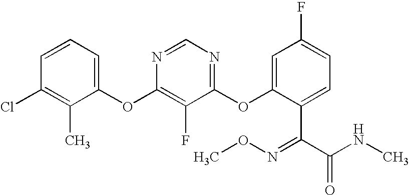 | 3.62 |
| 11 | 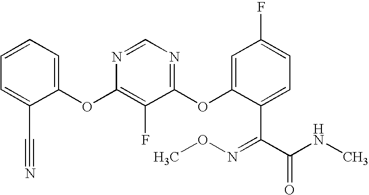 | 2.68 |
| 12 | 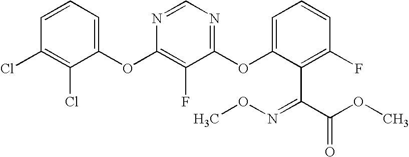 | 4.16 |
| 13 | 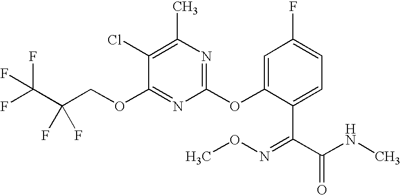 | 3.58 |
| 14 | 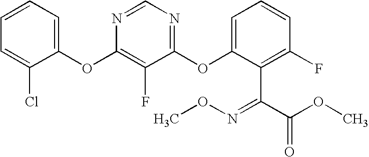 | 3.73 |
| 15 | 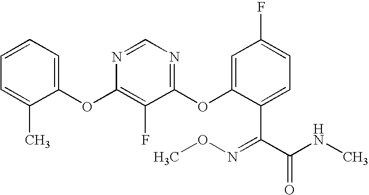 | 3.2 |
| 16 | 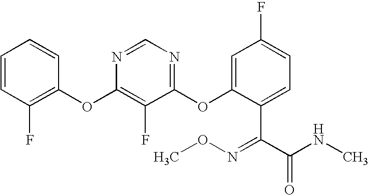 | 3.03 |
| 17 | 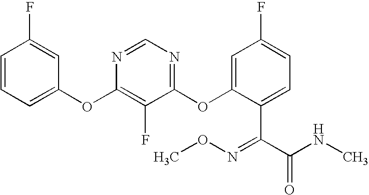 | 3.07 |
| 18 | 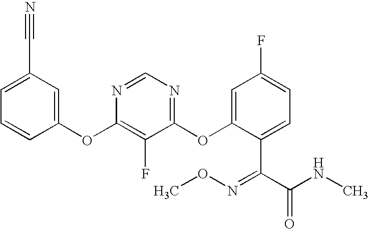 | 2.71 |
| 19 | 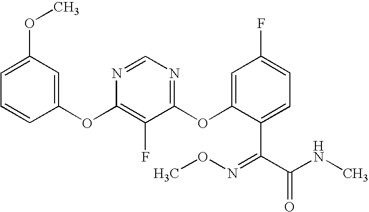 | 2.91 |
| 20 | 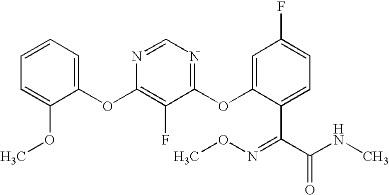 | 2.82 |
| 21 | 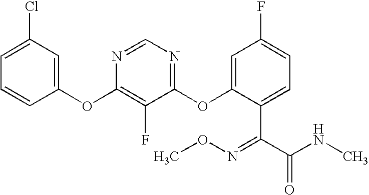 | 3.31 |
| 22 | 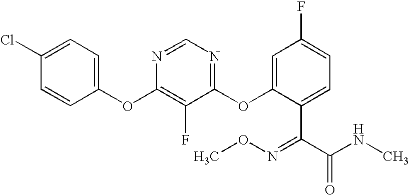 | 3.32 |
| 23 | 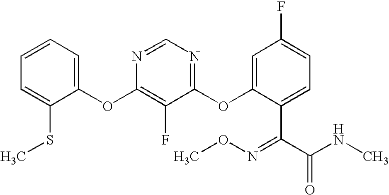 | 3.07 |
| 24 | 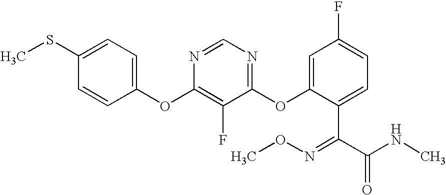 | 3.24 |
| 25 | 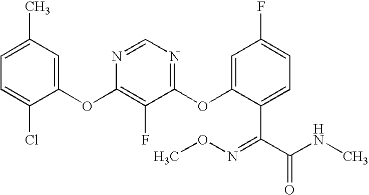 | 3.45 |
| 26 | 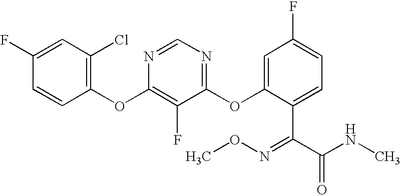 | 3.27 |
| 27 | 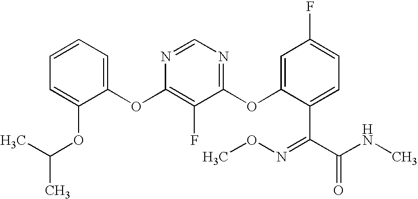 | 3.32 |
| 28 | 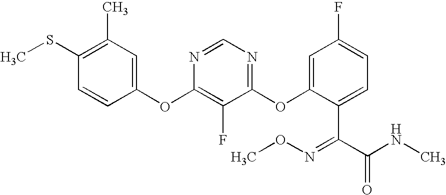 | 3.54 |
| 29 | 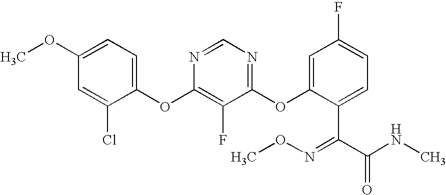 | 3.2 |
| 30 | 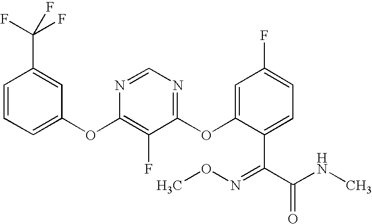 | 3.46 |
| 31 | 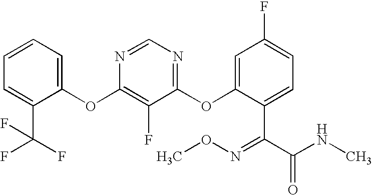 | 3.26 |
| 32 | 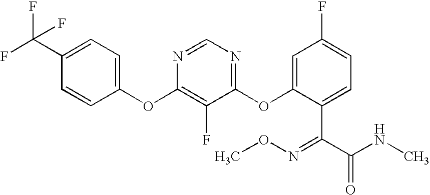 | 3.49 |
| 33 | 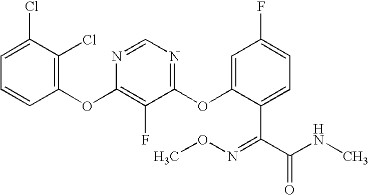 | 3.53 |
| 34 | 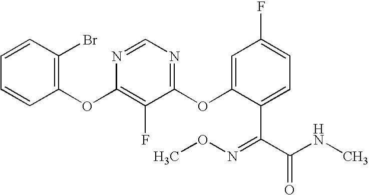 | 3.22 |
| 35 | 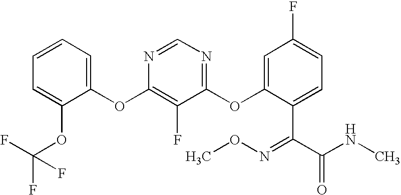 | 3.27 |
| 36 | 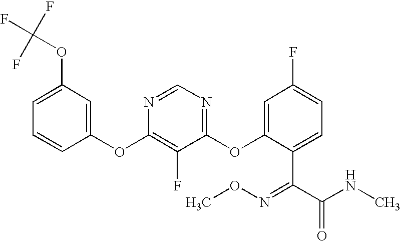 | 3.46 |
| 37 | 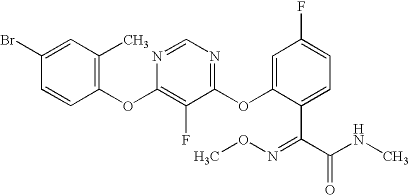 | 3.57 |
| 38 | 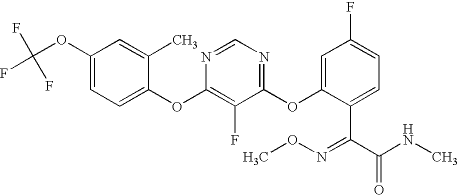 | 3.71 |
| 39 | 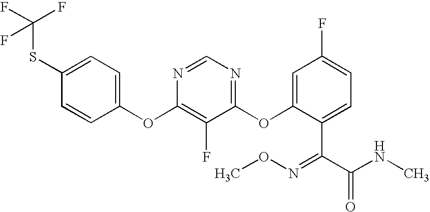 | 3.75 |
| 40 | 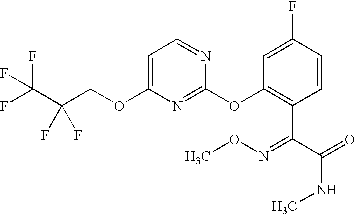 | 2.87 |
| 41 | 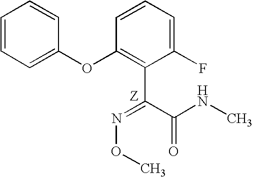 | 2.49 |
| 42 | 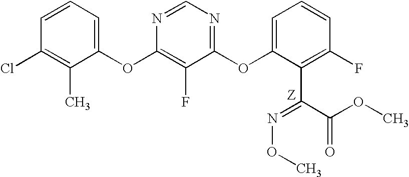 | 4.41 |
| 43 | 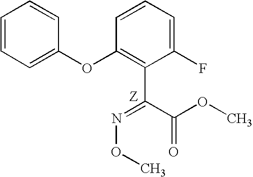 | 3.42 |
| 44 | 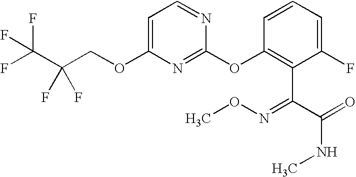 | 2.89 |
| 45 | 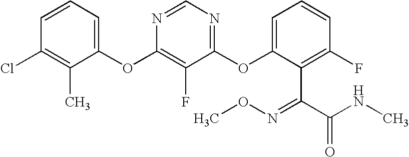 | 3.6 |
| 46 | 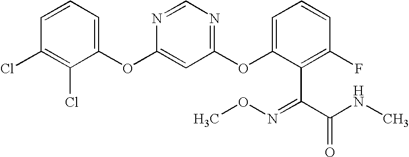 | 3.23 |
| 47 | 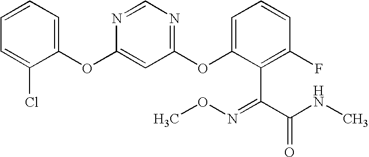 | 2.79 |
| 48 | 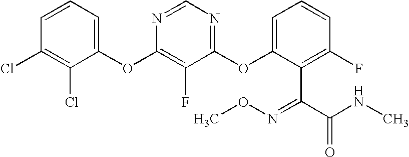 | 3.5 |
| 49 | 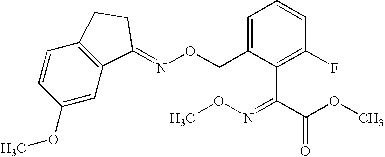 | 3.65 |
| 50 | 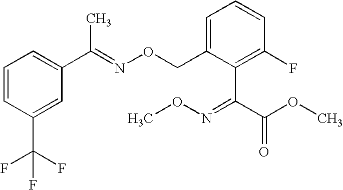 | 4.27 |
| 51 | 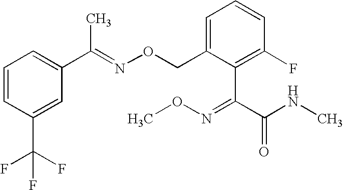 | 3.71 |
| 52 | 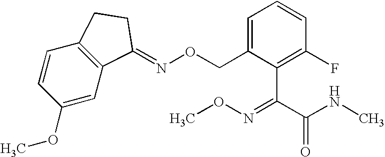 | 3.08 |
| 53 | 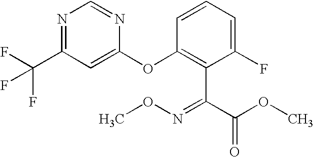 | 2.91 |
| 54 | 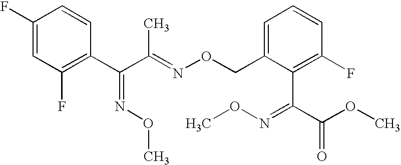 | 4.13 |
| 55 | 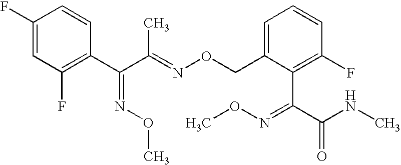 | 3.57 |
| 56 | 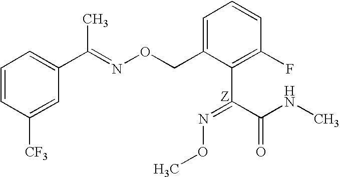 | 3.80 |
| 57 | 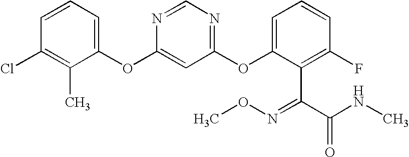 | 3.24 |
| 58 | 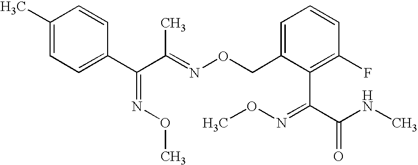 | 3.71 |
| 59 | 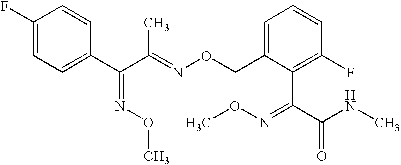 | 3.5 |

The logP values were determined in accordance with EEC Directive 79/831 AnnexeV.A8 by HPLC (gradient method, acetonitrile/0.1% aqueous phosphoric acid)

In the compounds listed above, the double bond of the methoxyimino-N-methylacetamide group or the methyl methoxyiminoacetate group is, unless stated otherwise, present in the E configuration.

### Preparation of the Precursors

### Compounds of the Formula (II)

### Example (II-1)

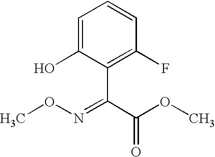 Process g)

A mixture of 47 g (0.282 mol) of methyl [2-fluoro-6-(tetrahydropyran-2-yloxy)phenyl]oxoacetate, 28,4 g (0.34 mol) of O-methylhydroxylamine hydrochloride and 250 ml of methanol is boiled under reflux for 18 hours. The solvent is then evaporated under reduced pressure, water is added to the residue and the mixture is extracted with ethyl acetate. The organic phase is dried over sodium sulphate and concentrated under reduced pressure. The residue is triturated with a mixture of petroleum ether and diisopropyl ether (about 1:1), and the crystalline product is filtered off with suction. This gives 26.9 g (42% of theory) of methyl (2-fluoro-6-hydroxyphenyl)methoxyiminoacetate of logP (pH 2)=1.67 (content owing to HPLC: 96%).

### Example (II-2)

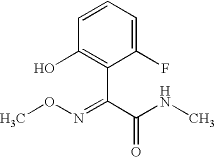 Process h)

A mixture of 11.4 g (0.05 mol) of methyl 2-(2-fluoro-6-hydroxyphenyl)-2-methoximinoacetate, 43 ml of a solution of methylamine in water (40%) and 150 ml of methanol is stirred at room temperature overnight. The solvent is then distilled off under reduced pressure, water is added to the residue and the mixture is acidified to pH 4 by addition of dilute hydrochloric acid. The precipitated product is filtered off with suction and washed with water. This gives 10.0 g (88.5% of theory) of 2-(2-fluoro-6-hydroxyphenyl)-2-methoxyimino-N-methylacetamide of log P (pH 2)=1.18.

### Example (II-3)

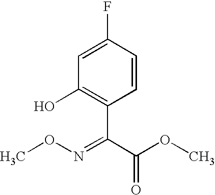 Process g)

A mixture of 7.5 g (0.038 mol) of methyl (4-fluoro-2-hydroxyphenyl)oxoacetate, 3.8 g (0.045 mol) of O-methylhydroxylamine hydrochloride and 70 ml of methanol is boiled under reflux for 18 hours. The solvent is then evaporated under reduced pressure, water is added to the residue and the mixture is extracted with ethyl acetate. The organic phase is dried over sodium sulphate and concentrated under reduced pressure. The residue is purified by column chromatography (petroleum ether/acetone =4:1). This gives 3.8 g (44% of theory) of methyl (4-fluoro-2-hydroxyphenyl)methoxyiminoacetate of logP (pH 2)=2.62.

### Example (II-4)

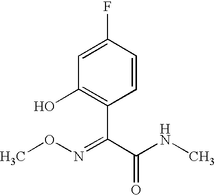 Process h)

A mixture of 2.7 g (0.012 mol) of methyl (4-fluoro-2-hydroxyphenyl)methoxyiminoacetate, 12 ml of a solution of methylamine in water (40%) and 60 ml of methanol is stirred at room temperature overnight. The solvent is then distilled off under reduced pressure, ethyl acetate is added to the residue and the solution is dried with sodium sulphate. The solvent is then distilled off under reduced pressure. This gives 2.7 g (quant.) of 2-(4-fluoro-2-hydroxyphenyl)-2-methoxyimino-N-methylacetamide of log P (pH 2)=1.56.

### Compounds of the Formula (XIII)

### Example (XIII-1)

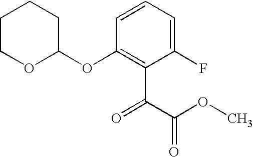 Process g)

At −75° C., 50 ml of a 2.5 M solution of butyllithium in hexane (0.12 mol) are added dropwise to a solution of 19.6 g (0.1 mol) of 2-(3-fluorophenoxy)tetrahydropyran (preparation see J.Amer.Chem.Soc. 119; 6; 1997; 1208-1216) in 100 ml of tetrahydrofuran, and the mixture is stirred at −75° C. for 1 hour. At −75° to −85° C., 50ml of a solution of 13 g (0.11 mol) of dimethyl oxalate in 100 ml of tetrahydrofuran are then added dropwise, and the mixture is stirred at −75° C. for 1 hour. The reaction mixture is then slowly allowed to warm to room temperature, and the solvent is then evaporated at 30° C. under reduced pressure. 300 ml of a saturated ammonium chloride solution are added to the residue, and the mixture is extracted with ethyl acetate. The organic phase is dried over sodium sulphate and concentrated under reduced pressure. The residue is triturated with petroleum ether, and the crystalline product is filtered off with suction. This gives 14.1 g (50% of theory) of methyl [2-fluoro-6-(tetrahydropyran-2-yloxy)phenyl]oxoacetate of logP (pH 2)=2.78 (content according to BPLC: 99%).

### Compounds of the Formula (XIV)

### Example (XIV-1)

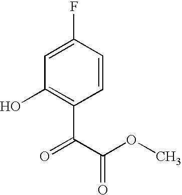 Process k)

At 30° C., 6.6 g (0.0535 mol) of methyl oxalyl chloride are added dropwise to a mixture of 21.4 g (0.161 g) of anhydrous aluminium chloride, 6 g (0.0535 mol) of 3-fluorophenol and 100 ml of dichloroethane. The reaction mixture is stirred at room temperature overnight and then poured into ice-water. The mixture is extracted twice with methylene chloride, the organic phase is dried and the solvent is evaporated under reduced pressure. This gives 8.9 g of a yellow oil which, according to HPLC, consists to 77% of methyl (4-fluoro-2-hydroxyphenyl)oxo-acetate. Log P (pH 2)=2.10.

### Compounds of the Formula (VIII)

### Example (VIII-1)

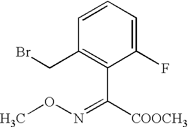 Process m)

A mixture of 21.6 g (0.096 mol) of methyl (2-fluoro-6-methylphenyl)methoxyiminoacetate, 13.7 g (0.048 mol) of 1,3-dibromo-5,5-dimethylhydantoin, 1 g of dibenzoyl peroxide and 250 ml of carbon tetrachloride is boiled under reflux for 7 hours. The reaction mixture is then filtered through Celite, and the solvent is evaporated under reduced pressure. This gives 29.4 g of a product which, according to HPLC analysis, consists to 67% of an E/Z mixture of methyl (2-bromomethyl-6-fluorophenyl)methoxyiminoacetate (log P (pH 2)=2.58 and 2.87).

### Compounds of the Formula (XVIII)

### Example (XVIII-1)

1. Methyl 2-(2-fluoro-6-methylphenyl)-2-methoximinoacetate 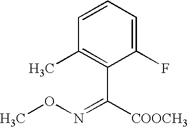
Process n)

A mixture of 19.6 g (0.1 mol) of methyl (2-fluoro-6-methylphenyl)oxoacetate, 10 g (0.12 mol) of O-methylhydroxylamine hydrochloride and 200 ml of methanol is boiled under reflux for 18 hours. The solvent is then evaporated under reduced pressure, water is added to the residue and the mixture is extracted with ethyl acetate. The organic phase is dried over sodium sulphate and concentrated under reduced pressure. This gives 22.5 g (quant.) of methyl (2-fluoro-6-methylphenyl)methoxyiminoacetate as an E/Z mixture of logP (pH 2)=2.43 and 2.74.

### Compounds of the Formula (XIX)

### Example (XIX-1)

2. Methyl 2-fluoro-6-methylphenylglyoxalate

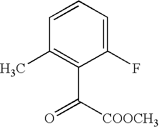

At room temperature, 21.1 g (0.207 mol) of acetic anhydride are added dropwise to a mixture of 33.7 g (0.207 mol) of 2-fluoro-6-methylbenzoyl cyanide in 300 ml of methyl tert-butyl ether, and the mixture is stirred at room temperature for 10 minutes. The mixture is then cooled to −15° C., and at this temperature HCl gas is introduced until the mixture is saturated. The reaction mixture is stirred at room temperature overnight, and 52 ml of methanol are then added. The mixture is stirred at room temperature for another 18 hours, and the solvent is then distilled off under reduced pressure. 250 ml of methyl tert-butyl ether are added to the residue, and the mixture is then extracted twice with in each case 200 ml of water. The organic phase is concentrated under reduced pressure, the residue is dissolved in 200 ml of methylene chloride and the solution is added dropwise to 42 ml of conc. hydrochloric acid. The mixture is stirred at room temperature overnight, and 100 ml of water are then added. The organic phase is separated off and dried over sodium sulphate, and the solvent is distilled off under reduced pressure. This gives 40.7 g (quant.) of methyl (2-fluoro-6-methylphenyl)oxoacetate of log P (pH 2)=2.44 (content according to HPLC: 93.5%).

### Compounds of the Formula (XX)

### Example (XX-1)

2-Fluoro-6-methylbenzoyl cyanide

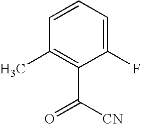

A mixture of 65 g (0.422 mol) of 2-fluoro-6-methylbenzoic acid (preparation see DE 3328494), 60.2 g (0.506 mol) of thionyl chloride, 0.1 ml of dimethylformamide and 250 ml of methylene chloride is boiled under reflux overnight. The solvent is then distilled off under reduced pressure. The residue (72 g) is heated to 130° C., and 43.5 g (0.438 mol) of trimethylsilyl cyanide are added dropwise. The trimethylchlorosilane that is formed is distilled off. After the end of the addition, the reaction mixture is heated at 180° C. for another 10 minutes, and the product is then distilled off under high vacuum. This gives 31.6 g (46.5% of theory) of 2-fluoro-6-methylbenzoyl cyanide of boiling point 80-83° C./1 mbar and log P (pH 2)=2.55.

### Use Examples

### Example A

Phytophthora test (tomato)/protective

| | |
|---|---|
| Solvent: | 24.5 parts by weight of acetone |
| | 24.5 parts by weight of dimethylacetamide |
| Emulsifier: | 1.0 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Phytophthora infestans. The plants are then placed in an incubation cabin at about 20° C. and 100% relative atmospheric humidity.

Evaluation is carried out 3 days after the inoculation. 0% means an efficacy which corresponds to that of the control, whereas an efficacy of 100% means that no infection is observed.

In this test, the compounds according to the invention listed in Examples (2, 7, 9, 12, 14, 15, 31, 33, 45, 46, 47, 48, 50, 51, 56, 57) exhibit an efficacy of 86% or more at an application rate of 100 g/ha.

### Example B

### Podosphaera test (apple)/protective

| | |
|---|---|
| Solvent: | 24.5 parts by weight of acetone |
| | 24.5 parts by weight of dimethylacetamide |
| Emulsifier: | 1.0 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the apple mildew pathogen Podosphaera leucotricha. The plants are then placed in a greenhouse at about 23° C. and about 70% relative atmospheric humidity.

Evaluation is carried out 10 days after the inoculation. 0% means an efficacy which corresponds to that of the control, whereas an efficacy of 100% means that no infection is observed.

In this test, the compounds according to the invention listed in Examples (1, 2, 5, 8, 9, 12, 14, 15, 31, 33, 35, 45, 46, 47, 48, 50, 51, 56, 57) exhibit an efficacy of 82% or more at an application rate of 100 g/ha.

### Example C

### Sphaerotheca test (cucumber)/protective

| | |
|---|---|
| Solvent: | 24.5 parts by weight of acetone |
| | 24.5 parts by weight of dimethylacetamide |
| Emulsifier: | 1.0 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Sphaerotheca fuliginea. The plants are then placed in a greenhouse at about 23° C. and about 70% relative atmospheric humidity.

Evaluation is carried out 7 days after the inoculation. 0% means an efficacy which corresponds to that of the control, whereas an efficacy of 100% means that no infection is observed.

In this test, the compounds according to the invention listed in Examples (1, 2, 7, 8, 9, 12, 14, 15, 31, 33, 35, 45, 46, 47, 48, 50, 51, 56, 57) exhibit an efficacy of98% or more at an application rate of 100 g/ha.

### Example D

### Venturia test (apple)/protective

Solvent: 24.5 parts by weight of acetone
   24.5 parts by weight of dimethylacetamide
Emulsifier: 1.0 parts by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous conidia suspension of the apple scab pathogen Venturia inaequalis and then remain in an incubation cabin at about 20° C. and 100% relative atmospheric humidity for 1 day.

The plants are then placed in a greenhouse at about 21° C. and a relative atmospheric humidity of about 90%.

Evaluation is carried out 10 days after the inoculation. 0% means an efficacy which corresponds to that of the control, whereas an efficacy of 100% means that no infection is observed.

In this test, the compounds according to the invention listed in Examples (1, 2, 7, 8, 9, 12, 14, 15, 31, 33, 35, 45, 46, 47, 48, 50, 51, 56, 57) exhibit an efficacy of 97% or more at an application rate of 100 g/ha.

### Example E

### Erysiphe test (barley)/protective

| | |
|---|---|
| Solvent: | 25 parts by weight of N,N-dimethylacetamide |
| Emulsifier: | 0.6 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate.

After the spray coating has dried on, the plants are dusted with spores of Erysiphe graminis f.sp. hordei.

The plants are placed in a greenhouse at a temperature of about 20° C. and a relative atmospheric humidity of about 80% to promote the development of mildew pustules.

Evaluation is carried out 7 days after the inoculation. 0% means an efficacy which corresponds to that of the control, whereas an efficacy of 100% means that no infection is observed.

In this test, the compounds according to the invention listed in Examples (1, 3, 4, 8, 10, 12, 14, 40, 41, 42, 43, 44, 50, 51) show an efficacy of 98% or more at an application rate of 500 g/ha.

### Example F

### Leptosphaeria nodorum test (wheat)/protective

| | |
|---|---|
| Solvent: | 25 parts by weight of N,N-dimethylacetamide |
| Emulsifier: | 0.6 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are sprayed with a spore suspension of Leptosphaeria nodorum. The plants remain in an incubation cabin at 20° C. and 100% relative atmospheric humidity for 48 hours.

The plants are placed in a greenhouse at a temperature of about 15° C. and a relative atmospheric humidity of 80%.

Evaluation is carried out 10 days after the inoculation 0% means an efficacy which corresponds to that of the control, whereas an efficacy of 100% means that no infection is observed.

In this test, the compounds according to the invention listed in Examples (2, 16, 22, 31, 45, 46, 47, 48, 57) exhibit an efficacy of 98% or more at an application rate of 500 g/ha.

### Example G

### Myzus test/broad beans

| | |
|---|---|
| Solvent: | 31 parts by weight of acetone |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with emulsifier-containing water to the desired concentration.

Broad bean seedlings (Vicia faba minor) which are infected by the green peach aphid (Myzus persicae) are dipped into a preparation of active compound of the desired concentration and placed into a plastic dish.

After the desired period of time, the kill in percent is determined. 100% means that all animals have been killed; 0% means that none of the animals have been killed.

In this test, for example, the compound of Preparation Example (56) effects, at an exemplary active compound concentration of 100 ppm, a kill of 100% after 6 days.

### Example H

### Spodoptera frugipereda test/synthetic feed

| | |
|---|---|
| Solvent: | 31 parts by weight of acetone |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with emulsifier-containing water to the desired concentration.

A stated amount of the preparation of active compound of the desired concentration is pipetted onto a standardized amount of synthetic feed. In 6 replications, in each case one larva (L3) of the army worm (Spodoptera frugiperda) is placed onto the feed.

After the desired period of time, the kill in % is determined. 100% means that all animals have been killed; 0% means that none of the animals have been killed.

In this test, the compound according to the invention listed in Example (1) exhibits a kill rate of from 98% or more at a concentration of active compound of 500 ppm.

1-18. (canceled)

19. A compound of formula (I) 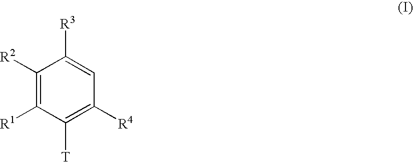
in which
R¹ represents one of the groups 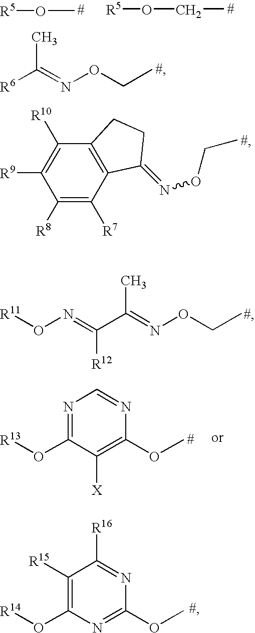
in which
# represents the point of attachment for R¹,
R⁵ represents optionally substituted aryl,
R⁶ represents optionally substituted aryl,
R⁷, R⁸, R⁹, and R¹⁰ are identical or different and independently of one another represent hydrogen, halogen, optionally substituted alkyl, or optionally substituted alkoxy,
R¹¹ represents optionally substituted alkyl,
R¹² represents optionally substituted alkyl, alkoxy, or aryl,
R¹³ represents optionally substituted alkyl or aryl,
R¹⁴ represents optionally substituted aryl,
R¹⁵ and R¹⁶ are identical or different and independently of one another represent hydrogen, halogen, or alkyl, and
X represents hydrogen or halogen,
R², R³, and R⁴ are identical or different and independently of one another represent hydrogen or halogen, with the proviso that at least one of the radicals R², R³, and R⁴ represents halogen, and
T represents a group 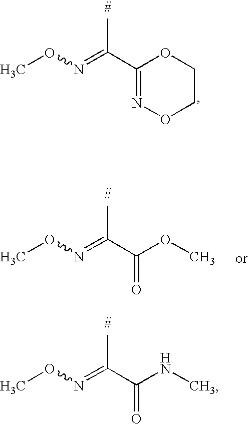
in which # represents the point of attachment for T.

20. A compound of formula (I) according to claim 19 in which
R¹ represents one of the groups 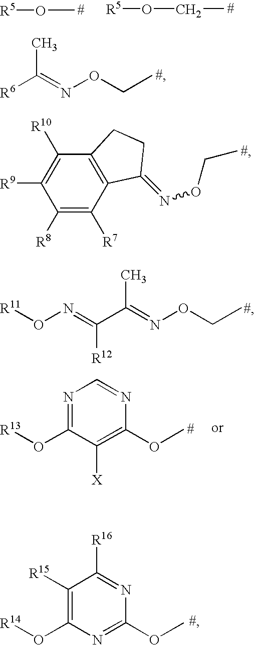
in which
# represents the point of attachment for R¹,
R⁵ represents optionally substituted phenyl,
R⁶ represents optionally substituted phenyl,
R⁷, R⁸, R⁹, and R¹⁰ are identical or different and independently of one another represent hydrogen, represent optionally halogen-substituted alkyl or optionally halogen-substituted alkoxy having in each case 1 to 4 carbon atoms, or represent fluorine, chlorine, bromine, or iodine,
R¹¹ represents optionally halogen-substituted alkyl having 1 to 4 carbon atoms,
R¹² represents optionally halogen-substituted alkyl or alkoxy having 1 to 6 carbon atoms or represents optionally substituted phenyl,
R¹³ represents optionally halogen-substituted alkyl having 1 to 6 carbon atoms or represents optionally substituted phenyl,
R¹⁴ represents optionally halogen-substituted alkyl having 1 to 6 carbon atoms,
R¹⁵ and R¹⁶ are identical or different and independently of one another represent hydrogen, fluorine, chlorine, bromine, or alkyl having 1 to 4 carbon atoms,
X represents hydrogen, fluorine, chlorine, bromine, or iodine,
R², R³, and R⁴ are identical or different and independently of one another represent hydrogen, fluorine, chlorine, bromine, or iodine, with the proviso that at least one of the radicals R², R³, and R⁴ represents fluorine, chlorine, bromine, or iodine, and
T represents a group 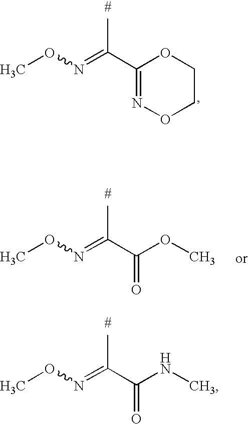
in which # represents the point of attachment for T,
where the optional substituents of the phenyl, phenoxy, and phenoxymethyl radicals are halogen, cyano, nitro, amino, hydroxyl, formyl, carboxyl, carbamoyl, or thiocarbamoyl; are straight-chain or branched alkyl, hydroxyalkyl, oxoalkyl, alkoxy, alkoxyalkyl, alkylthioalkyl, dialkoxyalkyl, alkylthio, alkylsulphinyl, or alkylsulphonyl having in each case 1 to 8 carbon atoms; are straight-chain or branched alkenyl or alkenyloxy having in each case 2 to 6 carbon atoms; are straight-chain or branched haloalkyl, haloalkoxy, haloalkylthio, haloalkylsulphinyl, or haloalkylsulphonyl having in each case 1 to 6 carbon atoms and 1 to 13 identical or different halogen atoms; are straight-chain or branched haloalkenyl or haloalkenyloxy having in each case 2 to 6 carbon atoms and 1 to 11 identical or different halogen atoms; are straight-chain or branched alkylamino or dialkylamino; are alkylcarbonyl, alkylcarbonyloxy, alkoxy-carbonyl, alkylaminocarbonyl, dialkylaminocarbonyl, arylalkylaminocarbonyl, dialkyl-aminocarbonyloxy, alkenylcarbonyl, or alkynylcarbonyl having 1 to 6 carbon atoms in the respective hydrocarbon chains; are cycloalkyl or cycloalkyloxy having in each case 3 to 6 carbon atoms; are doubly attached alkylene having 3 or 4 carbon atoms, oxyalkylene having 2 or 3 carbon atoms, or dioxyalkylene having 1. or 2 carbon atoms, each of which radicals is optionally mono- to tetrasubstituted by identical or different substituents selected from the group consisting of fluorine, chlorine, oxo, methyl, trifluoromethyl and ethyl; or are a group 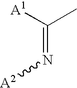
in which
A¹ represents hydrogen, hydroxyl, or alkyl having 1 to 4 carbon atoms or represents cycloalkyl having 1 to 6 carbon atoms, and
A² represents hydroxyl, amino, methylamino, phenyl, or benzyl, represents optionally cyano-, hydroxyl-, alkoxy-, alkylthio-, alkylamino-, dialkyl-amino-, or phenyl-substituted alkyl or alkoxy having 1 to 4 carbon atoms, or represents alkenyloxy or alkynyloxy having in each case 2 to 4 carbon atoms.

21. A compound of formula (I) according to claim 19 in which
R¹ represents one of the groups 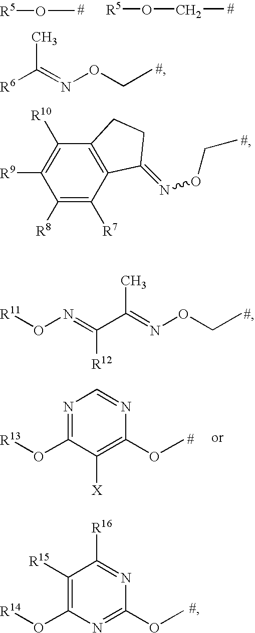
in which
# represents the point of attachment for R¹,
R⁵ represents optionally substituted phenyl,
R⁶ represents optionally substituted phenyl,
R⁷, R⁸, R⁹, and R¹⁰ are identical or different and independently of one another represent hydrogen, methyl, ethyl, trifluoromethyl, methoxy, ethoxy, trifluoromethoxy, fluorine, chlorine, bromine, or iodine,
R¹¹ represents methyl, ethyl, n- or i-propyl, or n-, i-, s-, or t-butyl,
R¹² represents methyl, ethyl, n- or i-propyl, n-, i-, s-, or t-butyl, methoxy, ethoxy, n- or i-propoxy, or n-, i-, s-, or t-butoxy or represents optionally substituted phenyl,
R¹³ represents methyl, ethyl, n- or i-propyl, n-, i-, s-, or t-butyl, pentyl, or hexyl or represents optionally substituted phenyl,
R¹⁴ represents optionally fluorine- or chlorine-substituted methyl, ethyl, n- or i-propyl, n-, i-, s-, or t-butyl, pentyl, or hexyl,
R¹⁵ and R¹⁶ are identical or different and independently of one another represent hydrogen, fluorine, chlorine, bromine, methyl, or ethyl, and
X represents hydrogen, fluorine, chlorine, bromine, or iodine,
R², R³, and R⁴ are identical or different and independently of one another represent hydrogen, fluorine, or chlorine, with the proviso that at least one of the radicals R², R³, and R⁴ represents fluorine or chlorine, and
T represents a group 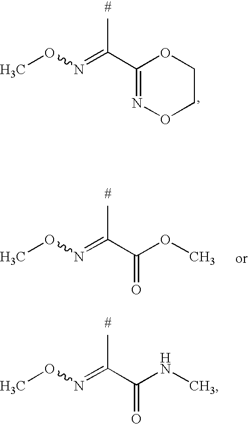
in which # represents the point of attachment for T,
where the optional substituents of the phenyl radicals are fluorine, chlorine, bromine, iodine, cyano, nitro, amino, hydroxyl, formyl, carboxy, carbamoyl, or thiocarbamoyl; are methyl, ethyl, n- or i-propyl, n-, i-, s-, or t-butyl, 1-, 2-, 3-, or neo-pentyl, 1-, 2-, 3-, or 4-(2-methylbutyl), 1-, 2-, or 3-hexyl, 1-, 2-, 3-, 4-, or 5-(2-methylpentyl), 1-, 2-, or 3-(3-methylpentyl), 2-ethylbutyl, 1-, 3-, or 4-(2,2-dimethylbutyl), 1-, or 2-(2,3-dimethylbutyl), hydroxymethyl, hydroxyethyl, 3-oxobutyl, methoxymethyl, or dimethoxymethyl; are methoxy, ethoxy, n- or i-propoxy, methoxymethyl, or ethoxy-methyl; are methylthio, ethylthio, n- or i-propylthio, methylsulphinyl, ethylsulphinyl, methylsulphonyl, ethylsulphonyl, methylthiomethyl, or ethylthiomethyl; are vinyl, allyl, 2-methylallyl, propen-1-yl, crotonyl, propargyl, vinyloxy, allyloxy, 2-methylallyloxy, propen-1-yloxy, crotonyloxy, or propargyloxy; are trifluoromethyl or trifluoroethyl; are difluoromethoxy, trifluoromethoxy, difluorochloromethoxy, trifluoroethoxy, difluoromethylthio, trifluoromethylthio, difluorochloromethylthio, trifluoromethylsulphinyl, or trifluoromethylsulphonyl; are methylamino, ethylamino, n- or i-propylamino, dimethylamino, or diethylamino; are acetyl, propionyl, methoxycarbonyl, ethoxycarbonyl, methylaminocarbonyl, ethylaminocarbonyl, dimethylaminocarbonyl, diethylaminocarbonyl, dimethylaminocarbonyloxy, diethylaminocarbonyloxy, benzylaminocarbonyl, acryloyl, or propioloyl; are cyclopentyl or cyclohexyl; are doubly attached propanediyl, ethyleneoxy, methylenedioxy, or ethylenedioxy, each of which is optionally mono- to tetra-substituted by identical or different substituents selected from the group consisting of fluorine, chlorine, oxo, methyl, and trifluoromethyl; or are a grouping 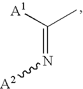
in which
A¹ represents hydrogen, methyl, or hydroxyl, and
A² represents hydroxyl, methoxy, ethoxy, amino, methylamino, phenyl, benzyl, or hydroxyethyl.

22. A process for preparing compounds of formula (I) according to claim 19 comprising
(a) reacting a hydroxyhalophenyl derivative of formula (II) 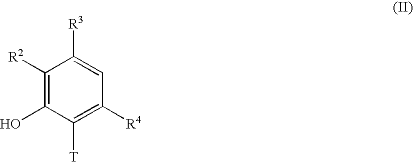
in which R², R³, R⁴, and T are as defined for formula (I) in claim 19, with a phenoxypyrimidine of formula (III) 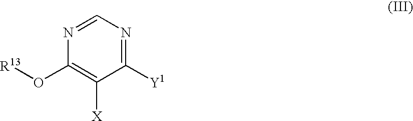
in which
R¹³ and X are as defined for formula (I) in claim 19, and
Y¹ represents halogen or with an alkoxypyrimidine of formula (IV) 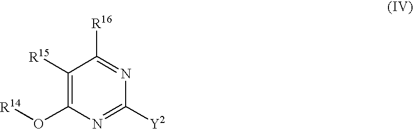
in which
R¹⁴, R¹⁵, and R¹⁶ are as defined for formula (I) in claim 19, and
Y² represents alkysulphonyl or arylsulphonyl, optionally in the presence of a diluent, optionally in the presence of an acid acceptor, and optionally in the presence of a catalyst,
or
(b) reacting a 4-phenoxypyrimidine of formula (V) 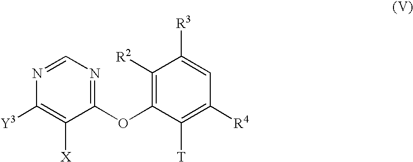
in which
R², R³, R⁴, T, and X are as defined for formula (I) in claim 19, and
Y³ represents halogen
with a phenol of formula (VI)
R^{1`3}—OH (VI)
in which R¹³ is as defined for formula (I) in claim 19, optionally in the presence of a diluent, optionally in the presence of an acid acceptor, and optionally in the presence of a catalyst, or that
(c) reacting a hydroxyhalophenyl derivative of the formula (II) with a diaryliodonium halide of formula (VII) 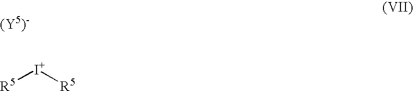
in which
R⁵ is as defined for formula (I) in claim 19, and
Y⁵ represents halogen, optionally in the presence of a diluent and optionally in the presence of an acid acceptor,
or
(d) reacting a 2-(2-halomethylphenyl)-2-methoxyimino derivative of formula (VIII) 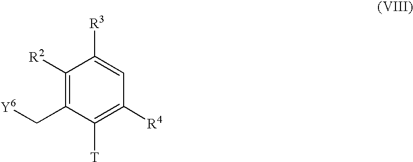
in which
R², R³, R⁴, and T are as defined for formula (I) in claim 19, and
Y⁶ represents halogen with an oxime of formula (IX) 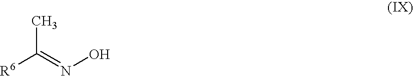
in which R⁶ is as defined for formula (I) in claim 19, or with a benzofuranone oxime of formula (X) 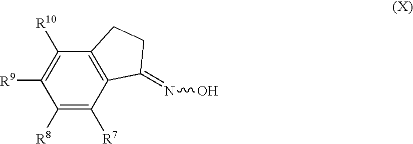
in which R⁷, R⁸, R⁹, and R¹⁰ are as defined for formula (I) in claim 19, or with a bisoxime of formula (XI) 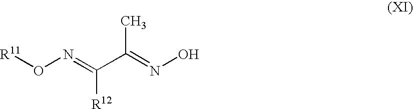
in which R¹¹ and R¹² are as defined for formula (I) in claim 19, or with a phenol of formula (XII)
R⁵—OH (XII)
in which R⁵ is as defined for formula (I) in claim 19, optionally in the presence of a diluent and optionally in the presence of an acid acceptor,
or
(e) reacting a halobenzene of formula (I-a) 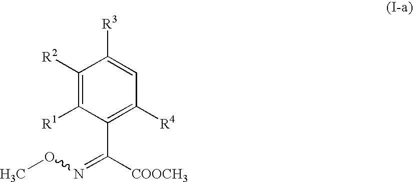
in which R¹, R², R³, and R⁴ are as defined for formula (I) in claim 19, with methylamine, optionally in the presence of a diluent,
or
(f) reacting a halobenzene of formula (I-a) in a first step with hydroxylamine or a salt thereof, optionally in the presence of an acid acceptor and optionally in the presence of a diluent, and without isolation, reacting the product of the first step in a second step with dibromoethane, optionally in the presence of an acid acceptor and optionally in the presence of a diluent.

23. A compound of formula (I-a) 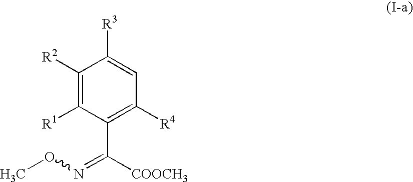
in which
R¹ represents one of the groups 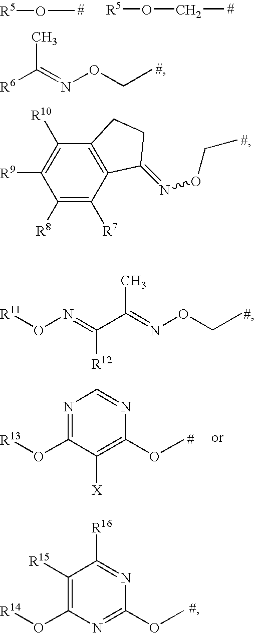
in which
# represents the point of attachment for R¹,
R⁵ represents optionally substituted aryl,
R⁶ represents optionally substituted aryl,
R⁷, R⁸, R⁹, and R¹⁰ are identical or different and independently of one another represent hydrogen, halogen, optionally substituted alkyl, or optionally substituted alkoxy,
R¹¹ represents optionally substituted alkyl,
R¹² represents optionally substituted alkyl, alkoxy, or aryl,
R¹³ represents optionally substituted alkyl or aryl,
R¹⁴ represents optionally substituted aryl,
R¹⁵ and R¹⁶ are identical or different and independently of one another represent hydrogen, halogen, or alkyl, and
X represents hydrogen or halogen, and
R², R³, and R⁴ are identical or different and independently of one another represent hydrogen or halogen, with the proviso that at least one of the radicals R², R³, and R⁴ represents halogen.

24. A compound of formula (II) 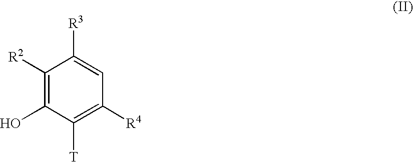
in which
R², R³, and R⁴ are identical or different and independently of one another represent hydrogen or halogen, with the proviso that at least one of the radicals R², R³, and R⁴ represents halogen, and
T represents a group 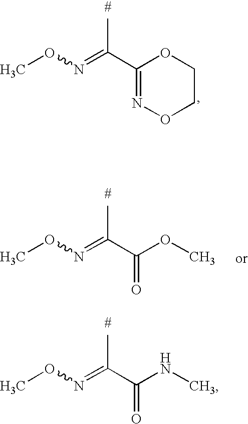
in which # represents the point of attachment for T.

25. A compound of formula (V) 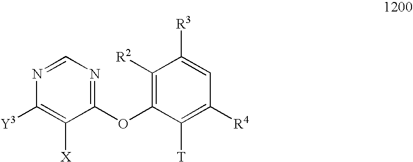
in which
R², R³, and R⁴ are identical or different and independently of one another represent hydrogen or halogen, with the proviso that at least one of the radicals R², R³, and R⁴ represents halogen,
T represents a group 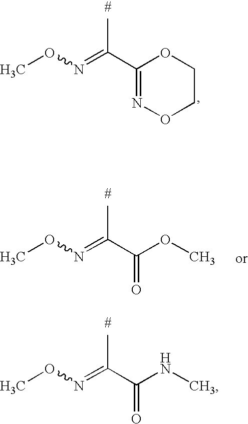
in which # represents the point of attachment for T,
X represents hydrogen or halogen, and
Y³ represents halogen.

26. A compound of formula (VIII) 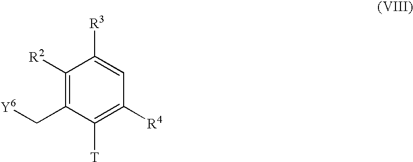
in which
R², R³, and R⁴ are identical or different and independently of one another represent hydrogen or halogen, with the proviso that at least one of the radicals R², R³, and R⁴ represents halogen,
T represents a group 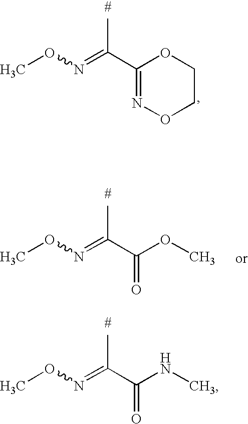
in which # represents the point of attachment for T, and
Y⁶ represents halogen.

27. A compound of formula (XIV) 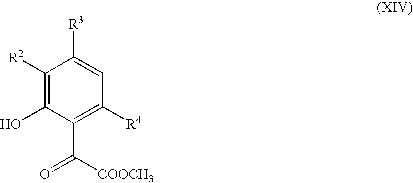
in which R², R³, and R⁴ are identical or different and independently of one another represent hydrogen or halogen, with the proviso that at least one of the radicals R², R³, and R⁴ represents halogen.

28. A compound of formula (XIII) 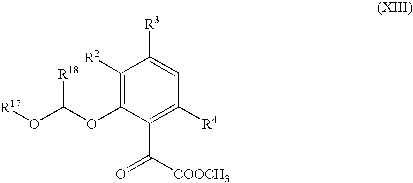
in which
R², R³, and R⁴ are identical or different and independently of one another represent hydrogen or halogen, with the proviso that at least one of the radicals R², R³, and R⁴ represents halogen,
R¹⁷ represents alkyl, and
R¹⁸ represents hydrogen or alkyl, or
R¹⁷ and R¹⁸ together with the atoms to which they are attached form a six-membered ring.

29. A compound of formula (XVIII) 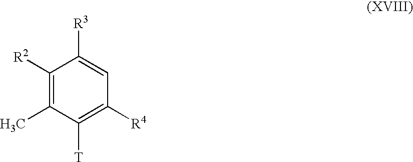
in which
R², R³, and R⁴ are identical or different and independently of one another represent hydrogen or halogen, with the proviso that at least one of the radicals R², R³, and R⁴ represents halogen, and
T represents a group 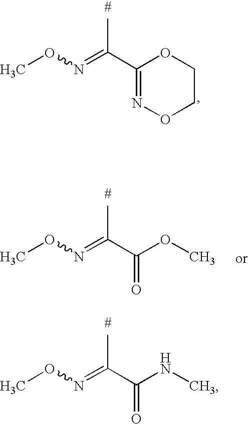
in which # represents the point of attachment for T.

30. A compound of formula (XVIIIa) 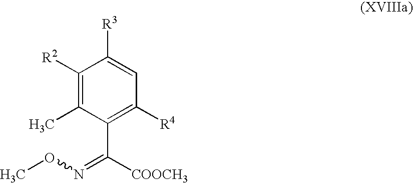
in which R², R³, and R⁴ are identical or different and independently of one another represent hydrogen or halogen, with the proviso that at least one of the radicals R², R³, and R⁴ represents halogen.

31. A compound of formula (XIX) 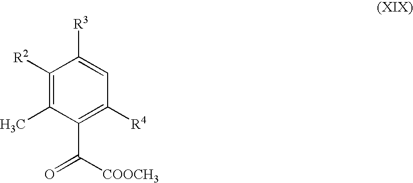
in which R², R³, and R⁴ are identical or different and independently of one another represent hydrogen or halogen, with the proviso that at least one of the radicals R², R³, and R⁴ represents halogen.

32. A compound of formula (XX) 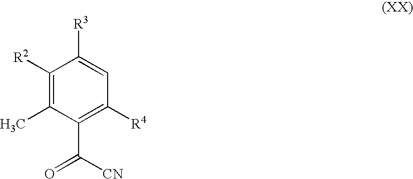
in which R², R³, and R⁴ are identical or different and independently of one another represent hydrogen or halogen, with the proviso that at least one of the radicals R², R³, and R⁴ represents halogen.

33. A composition for controlling harmful organisms comprising one or more compounds according to claim 19, one or more extenders and/or carriers, and, optionally, surfactants.

34. A method for controlling harmful organisms comprising allowing an effective amount of one or more compounds according to claim 19 to act on harmful organisms and/or their habitat.

35. A method for controlling harmful organisms comprising allowing an effective amount of one or more compositions according to claim 33 to act on harmful organisms and/or their habitat.

36. A process for preparing a composition according to claim 33 comprising combining one or more compounds according to claim 19 with one or more extenders and/or carriers and/or surfactants.
